(19) United States
(12) Patent Application Publication
(10) Pub. No.: US 2024/0180852 A1
(43) Pub. Date: Jun. 6, 2024

(54) TREATMENT FOR MALIGNANT PLEURAL EFFUSION IN HUMANS

(71) Applicant: Signpath Pharma, Inc., Sandy, UT (US)

(72) Inventors: Ashleigh Hocking, Bedford Park (AU); Peter P. Sordillo, New York, NY (US); Alexandra L. Farrall, Bedford Park (AU); Sonya Klebe, Bedford Park (AU)

(21) Appl. No.: 18/263,649

(22) PCT Filed: Mar. 2, 2022
(86) PCT No.: PCT/US22/18504

§ 371 Date: Jul. 31, 2023

Related U.S. Application Data
(60) Provisional application No. 63/156,090, filed on Mar. 3, 2021.

(51) Int. Cl. A61K 31/12; A61K 9/127; A61K 31/357

(52) U.S. Cl. CPC A61K 31/12; A61K 9/127; A61K 31/357

(57) ABSTRACT

The present invention includes method for treating malignant pleural effusion in a human patient comprising: administering to the human patient a therapeutically effective amount of a liposomal curcumin or liposomal curcuminoids intrapleurally, wherein the therapeutically effective amount is sufficient to reduce or treat the malignant pleural effusion.

### CROSS-REFERENCE TO RELATED APPLICATIONS

This application claims priority to U.S. Provisional Application No. 63/156,090 filed on 3 Mar. 2021, the contents of each of which are incorporated by reference herein.

### TECHNICAL FIELD OF THE INVENTION

The present invention relates in general to the field of treating malignant pleural effusion, and more particularly, to compositions and methods for the treatment of malignant pleural effusion in humans.

### STATEMENT OF FEDERALLY FUNDED RESEARCH

None.

### BACKGROUND OF THE INVENTION

Without limiting the scope of the invention, its background is described in connection with malignant pleural effusion.

Pleural effusion can be generally divided into two categories: transudative and exudative pleural effusion. Transudative pleural effusion is commonly caused by systemic disease or certain organ disease, such as heart failure, cirrhosis or kidney failure. Typically, pleural effusion happens on both sides of the chest simultaneously. Exudative pleural effusion is usually caused by lung disease, such as pneumonia, pulmonary tuberculosis and the like, or by cancer, such as lung cancer, breast cancer and the like; and pleural effusion happens on the one side of the chest mostly.

Common symptoms of pleural effusion include cough, dyspnea, chest pain, loss of breath, and/or weakened respiration. Severe cases will be accompanied with trachea to the contralateral displacement, or need of an oxygen generator to assist in breath.

Patients with non-malignant pleural effusion are often treated by discharging the fluid and treating the primary disease simultaneously. In patients with malignant pleural effusion, it is difficult to cure tumors. Drainage is a common way to discharge the fluid in the chest, and it can achieve the goal of discharging the fluid quickly, however, drainage alone does not treat the underlying malignancy. As such, drainage is merely an adjuvant therapy because the patient continually produces the fluid in the chest. Further, continuous drainage often causes irreversible damages to the patient.

Therefore, for patients with malignant pleural effusion, it is desired to treat pleural effusion with a safer or long-term treatment. What is needed are novel compositions and methods for treating and/or reducing malignant pleural effusion.

### SUMMARY OF THE INVENTION

In one embodiment, the present invention includes a method for treating malignant pleural effusion in a human patient comprising: administering to the human patient a therapeutically effective amount of a liposomal curcumin or liposomal curcuminoids intrapleurally, wherein the therapeutically effective amount is sufficient to reduce or treat the malignant pleural effusion. In one aspect, a pleural space or cavity is adjacent to at least one of the lung, heart, kidney, or liver. In another aspect, the liposomal curcumin or liposomal curcuminoids are administered at 50, 100, 150, 200, 250, 300, 325 or 350 mg/m². In another aspect, the liposomal curcumin or liposomal curcuminoids are administered at a dose of 125, 150, 200, 250, 300, 325, 350, 400, 450, 500, or 600 mg/m². In another aspect, the liposomal curcumin or liposomal curcuminoids are administered at a dose of 100 to 600 mg/m², over 2 to 6 hours. In another aspect, the liposomal curcumin or liposomal curcuminoids are administered at a dose of greater than 300 to 600 mg/m², over 2 to 4 hours. In another aspect, the liposomal curcumin or liposomal curcuminoids are administered at a dose of greater than 300 to 600 mg/m², over 2 or less. In another aspect, the liposomal curcumin or liposomal curcuminoids are administered for 2, 3, 4, 5, 6, or 7 hours. In another aspect, the liposomal curcumin or liposomal curcuminoids are administered at a dose of greater than 300 mg/m² over 2, 3, 4, 5, 6, 7, or 8 hours. In another aspect, the curcumin or curcuminoids are chemically synthesized curcumin or curcuminoids. In another aspect, the liposome comprises at least one of: 1,2-dimyristoyl-sn-glycero-3-phosphocholine (DMPC), 1,2-dimyristoyl-sn-glycero-3-phosphorylglycerol (DMPG), DMPC/DMPG, lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, or combinations thereof. In another aspect, the liposomal curcumin or curcuminoids are curcumin/curcuminoid:liposome complex, wherein the curcumin comprises between 2 to 9 weight percent of the curcumin/curcuminoid:liposome complex, wherein the curcumin is at least one of natural or synthetic curcumin and wherein the curcumin/curcuminoid:liposome complex has a ratio of curcumin to lipid (weight to weight) of 1:7.5 to 1:10, wherein the lipid combination is selected from: DMPC; DMPC:Chol 9:1; DMPC:DMPG 9:1; DMPC:Chol:DMPG 8:1:1; DPPC:DMPG 9:1; DPPC:Chol:DMPG 8:1:1; DMPC:DSPE-PEG-2000 95:5; DMPC:Chol:DSPE-PEG-2000 90:10:05; DMPC/DMPG 7:3; DPPC/DMPG 7:3; or DPPC/DMPG 9:1. In another aspect, the liposome comprises lipids of Formula I:

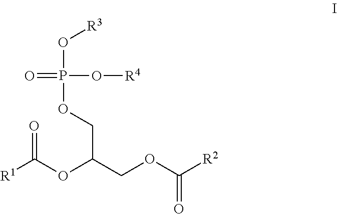

wherein, R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds; R³ is

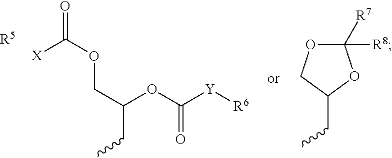

R⁴ is H or a pharmaceutically acceptable cation, wherein incorporation of said pharmaceutically acceptable cation results in a salt, e.g., a monomeric salt, a dimeric salt, a trimeric salt, or a multimeric salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. In another aspect, the compound of Formula I is selected from one or more of:

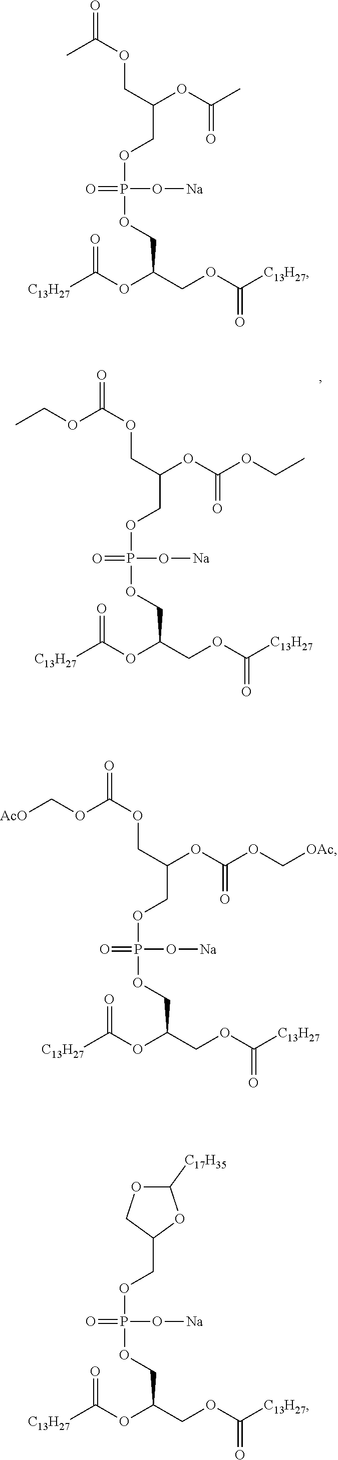

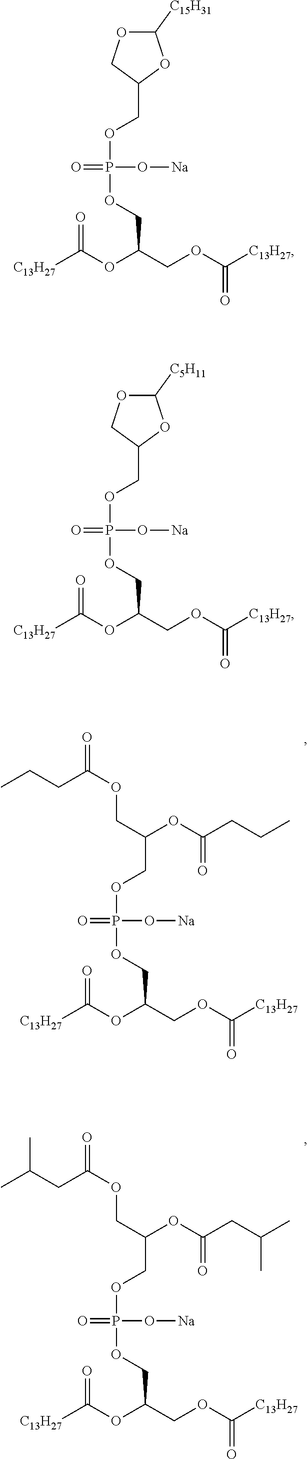

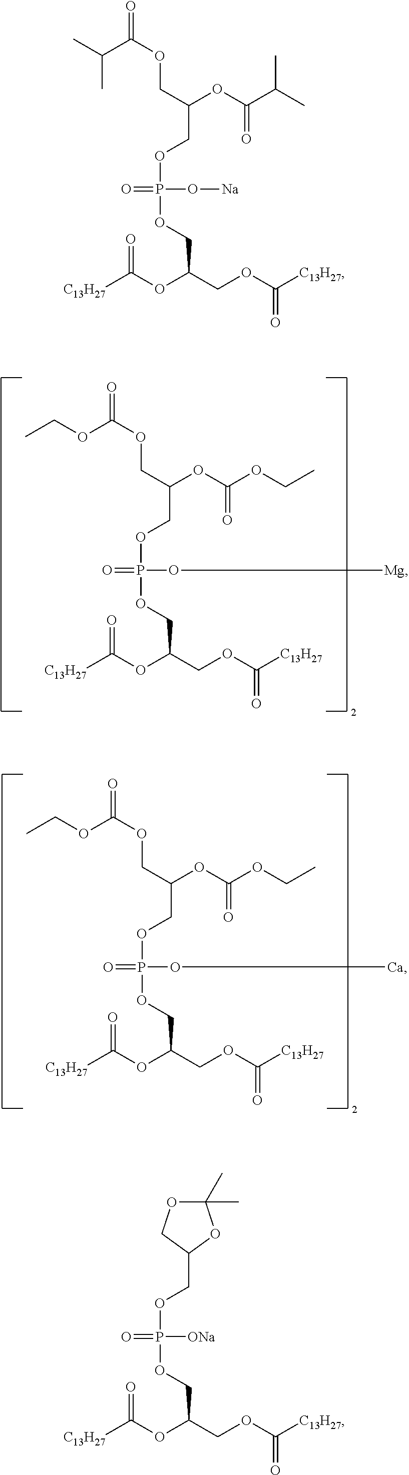

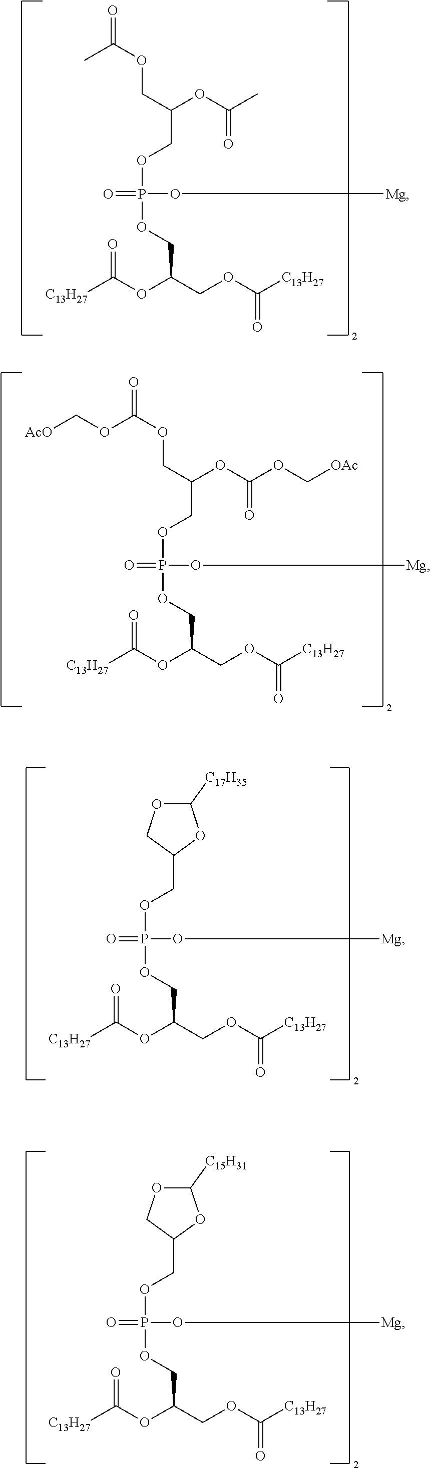

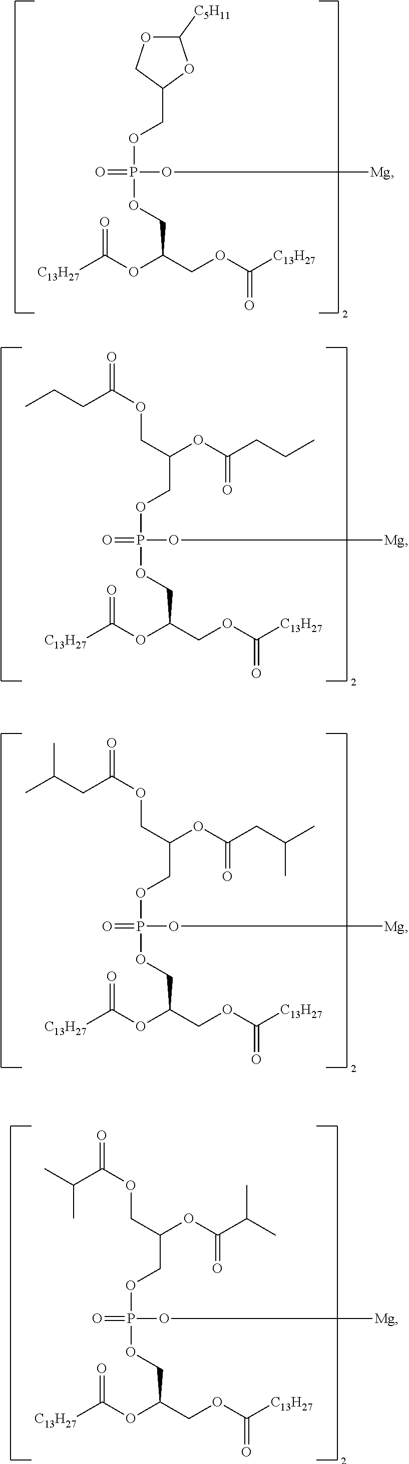

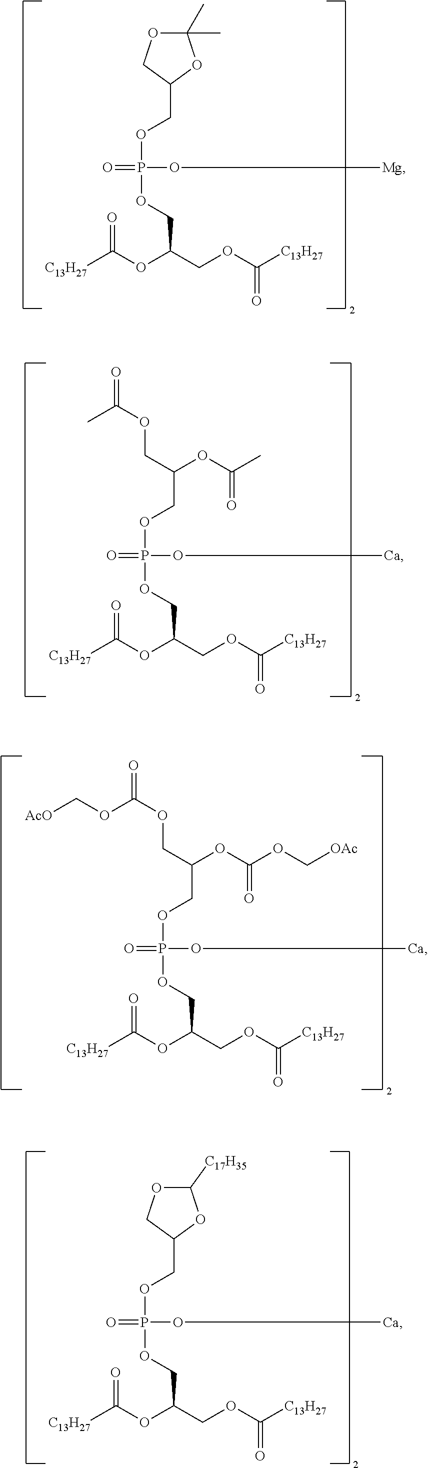

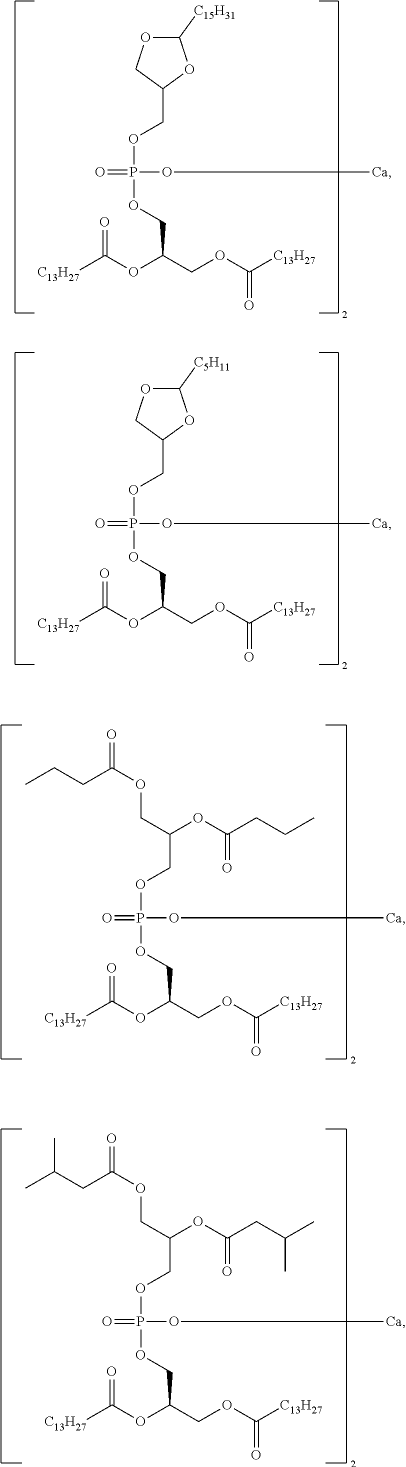

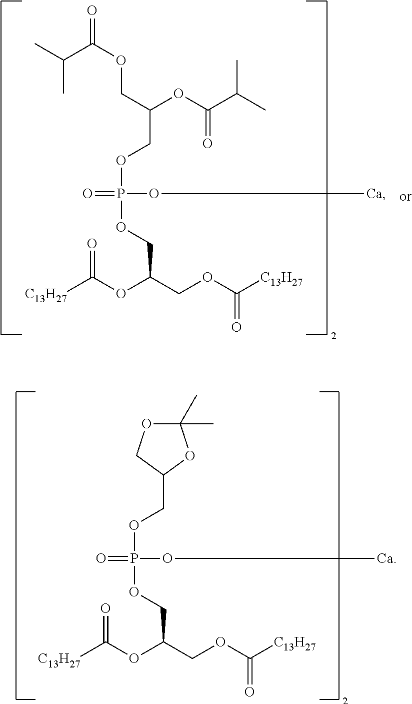

In another aspect, the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject. In another aspect, the composition further comprises a curcumin, or a synthetic curcumin that is 85, 86, 87, 88, 89, 90, 91, 92, 93, 94, 95 or 96% pure diferuloylmethane. In another aspect, the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

In another embodiment, the present invention includes a method for treating a malignant pleural effusion in a human patient comprising: identifying a human patient in need of a treatment for the malignant pleural effusion; and administering to the human patient a therapeutically effective amount of a liposomal curcumin or liposomal curcuminoids in a pleural space or cavity that is adjacent to at least one of the lung, heart, kidney, or liver, sufficient to reduce or treat the malignant pleural effusion. In one aspect, the liposomal curcumin or liposomal curcuminoids are administered at 50, 100, 150, 200, 250, 300, 325 or 350 mg/m². In another aspect, the liposomal curcumin or liposomal curcuminoids are administered at a dose of 125, 150, 200, 250, 300, 325, 350, 400, 450, 500, or 600 mg/m². In another aspect, the liposomal curcumin or liposomal curcuminoids are administered at a dose of 100 to 600 mg/m², over 2 to 6 hours. In another aspect, the liposomal curcumin or liposomal curcuminoids are administered at a dose of greater than 300 to 600 mg/m², over 2 to 4 hours. In another aspect, the liposomal curcumin or liposomal curcuminoids are administered at a dose of greater than 300 to 600 mg/m², over 2 or less. In another aspect, the liposomal curcumin or liposomal curcuminoids are administered for 2, 3, 4, 5, 6, or 7 hours. In another aspect, the liposomal curcumin or liposomal curcuminoids are administered at a dose of greater than 300 mg/m² over 2, 3, 4, 5, 6, 7, or 8 hours. In another aspect, the curcumin or curcuminoids are chemically synthesized curcumin or curcuminoids. In another aspect, the liposome comprises at least one of: 1,2-dimyristoyl-sn-glycero-3-phosphocholine (DMPC), 1,2-dimyristoyl-sn-glycero-3-phosphorylglycerol (DMPG), DMPC/DMPG, lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, or combinations thereof. In another aspect, the liposomal curcumin or curcuminoids are curcumin/curcuminoid:liposome complex, wherein the curcumin comprises between 2 to 9 weight percent of the curcumin/curcuminoid:liposome complex, wherein the curcumin is at least one of natural or synthetic curcumin and wherein the curcumin/curcuminoid:liposome complex has a ratio of curcumin to lipid (weight to weight) of 1:7.5 to 1:10, wherein the lipid combination is selected from: DMPC; DMPC:Chol 9:1; DMPC:DMPG 9:1; DMPC:Chol:DMPG 8:1:1; DPPC:DMPG 9:1; DPPC:Chol:DMPG 8:1:1; DMPC:DSPE-PEG-2000 95:5; DMPC:Chol:DSPE-PEG-2000 90:10:05; DMPC/DMPG 7:3; DPPC/DMPG 7:3; or DPPC/DMPG 9:1. In another aspect, the liposome comprises lipids of Formula I:

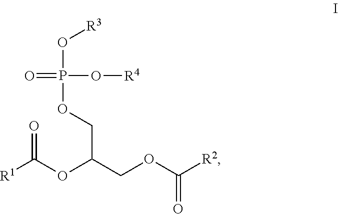

wherein, R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds; R³ is

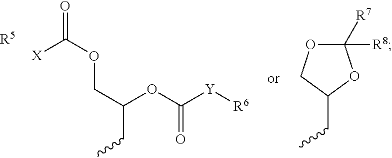

R⁴ is H or a pharmaceutically acceptable cation, wherein incorporation of said pharmaceutically acceptable cation results in a salt, e.g., a monomeric salt, a dimeric salt, a trimeric salt, or a multimeric salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. In another aspect, the compound of Formula I is selected from one or more of:

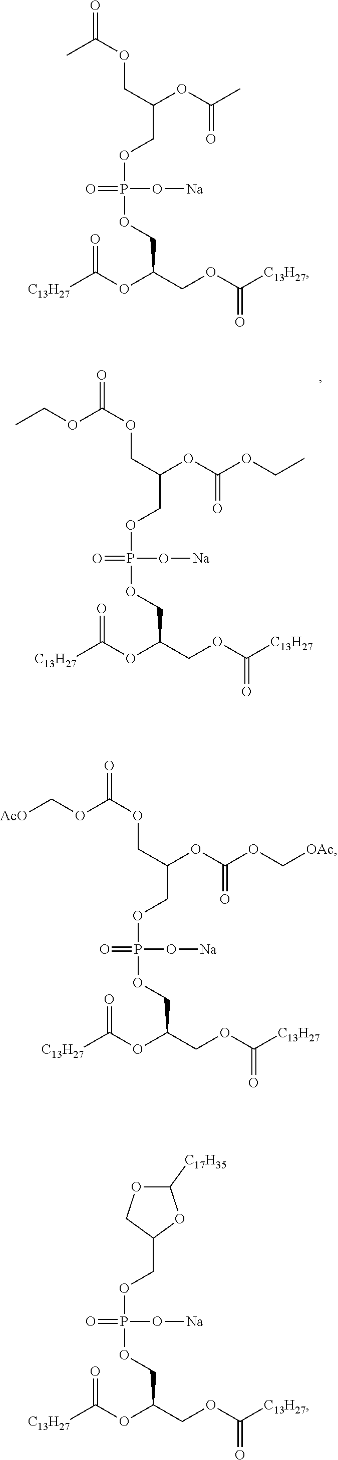

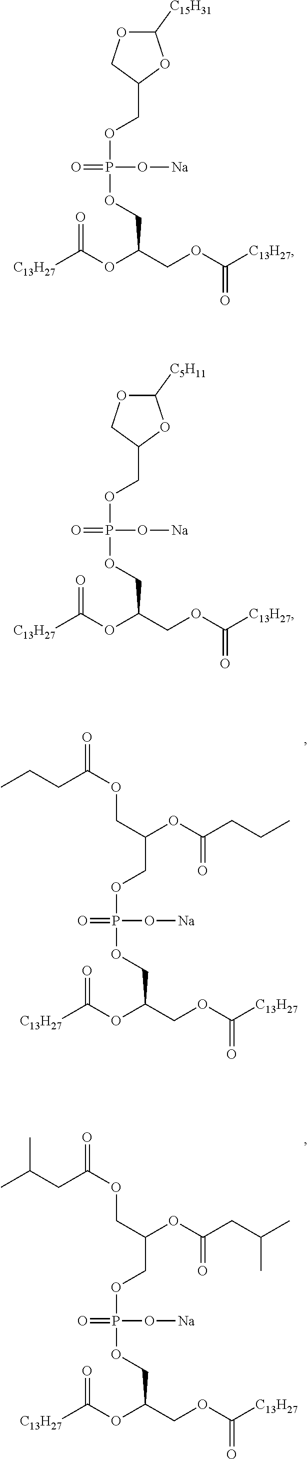

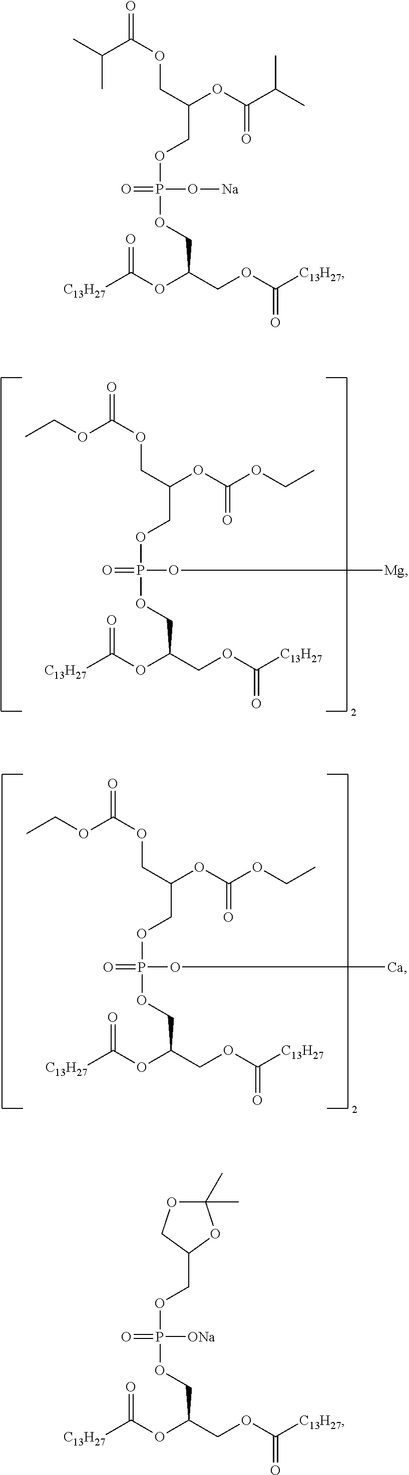

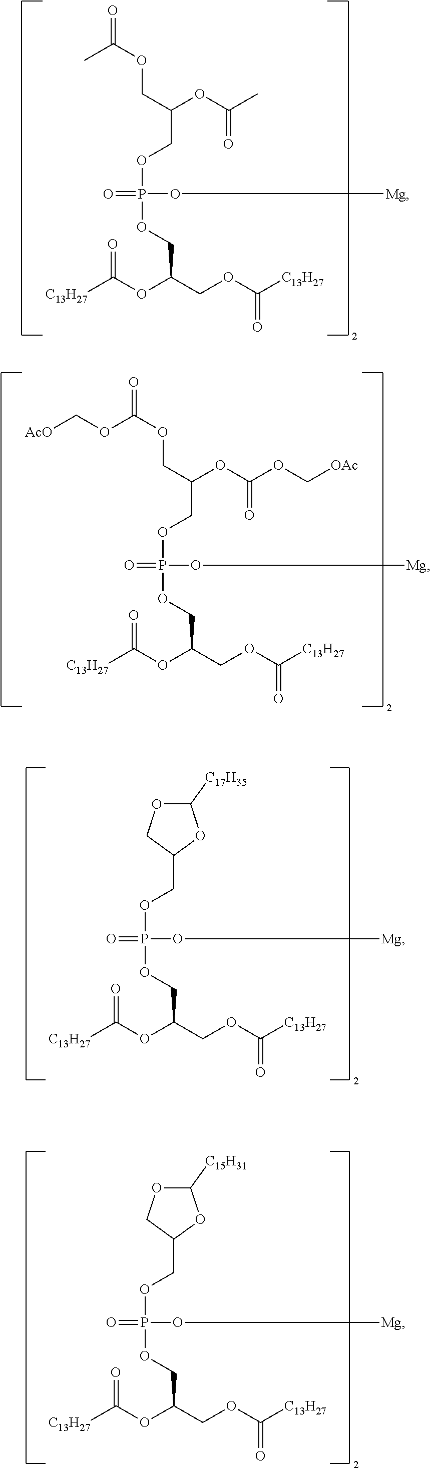

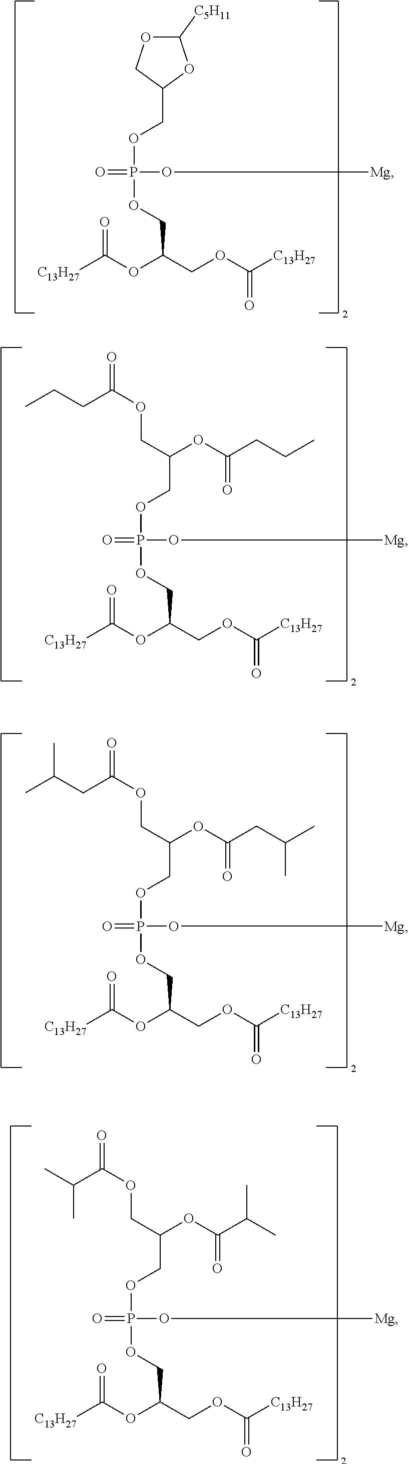

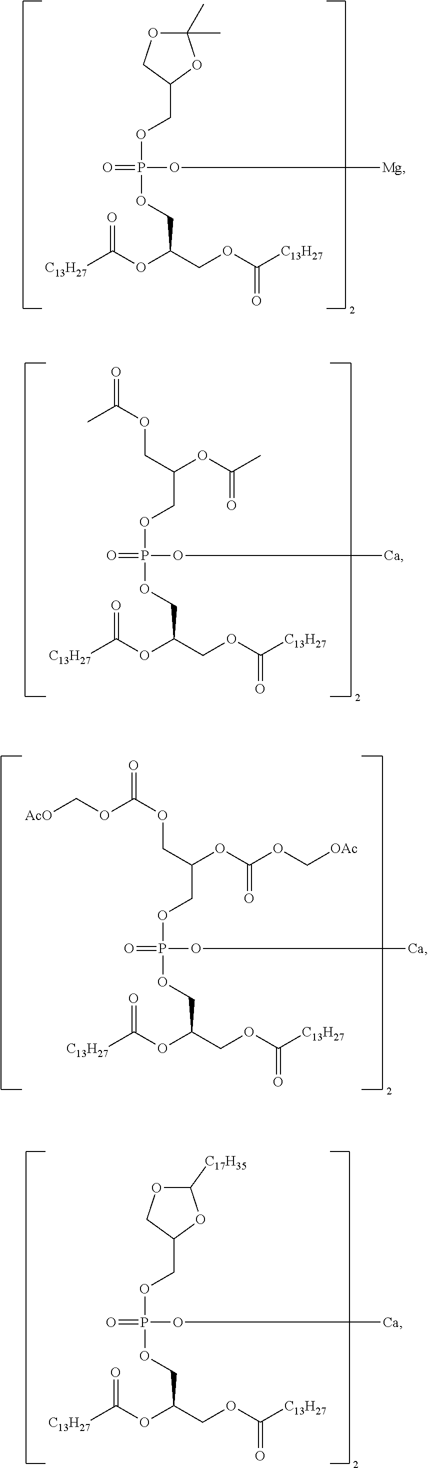

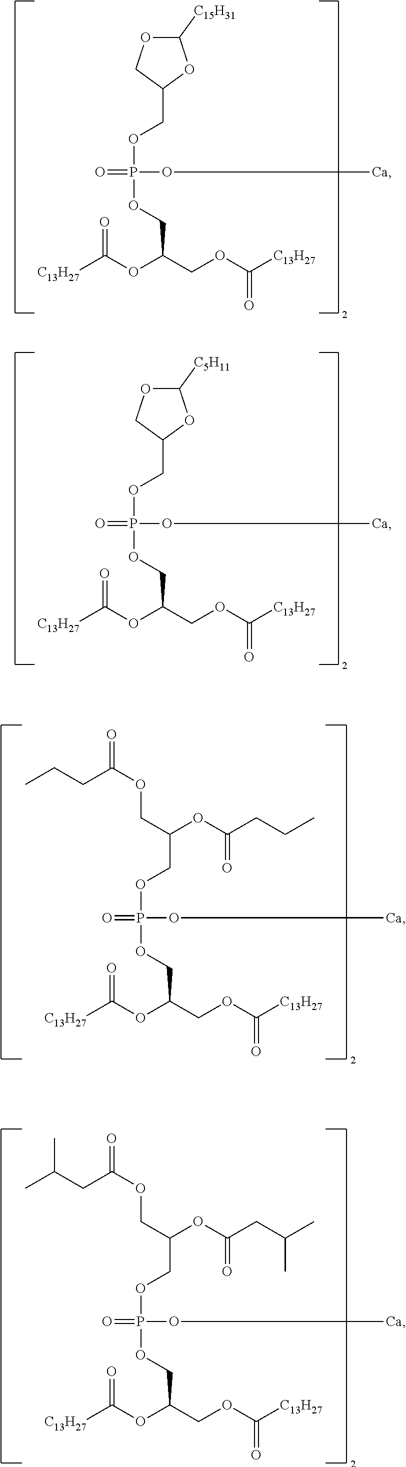

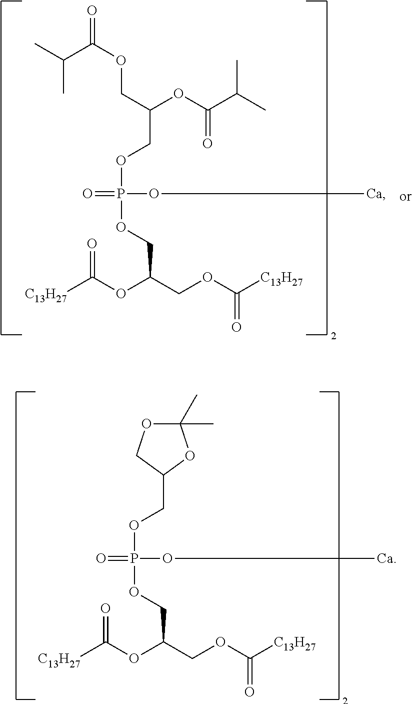

In another aspect, the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject. In another aspect, the composition further comprises a curcumin, or a synthetic curcumin that is 85, 86, 87, 88, 89, 90, 91, 92, 93, 94, 95 or 96% pure diferuloylmethane. In another aspect, the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a” “an” and “the” are not intended to refer to only a singular entity, but include the general class of which a specific example may be used for illustration. The terminology herein is used to describe specific embodiments of the invention, but their usage does not delimit the invention, except as outlined in the claims.

This study evaluates the tolerability and pharmacokinetic profiles of a single dose of liposomal curcumin, administered via an existing tunnelled indwelling pleural catheter (TIPC) directly to the tumour site in individuals with diagnoses of malignant pleural effusion. A maximum tolerated dose is determined for liposomal curcumin administered via this method.

As used herein, the term “in vivo” refers to being inside the body. The term “in vitro” used as used in the present application is to be understood as indicating an operation carried out in a non-living system.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology); or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the terms “effective amount” or “therapeutically effective amount” described herein means the amount of the subject compound that will elicit the biological or medical response of a tissue, system, animal or human that is being sought by the researcher, veterinarian, medical doctor or other clinician.

As used herein, the terms “administration of” or “administering a” compound as used herein should be understood to mean providing a compound of the invention to the individual in need of treatment in a form that can be introduced into that individual's body in a therapeutically useful form and therapeutically useful amount, including, but not limited to: tablets, capsules, syrups, suspensions, creams, jellies, powders, or patches adapted or formulated for intrapleural administration.

As used herein the term “intrapleural administration” includes injection and other modes of intrapleural administration, wherein the pleural areas include a pleural space or cavity adjacent to the lungs, kidneys, liver, and heart.

As used herein, the term “pharmaceutically acceptable” as used herein to describe a carrier, diluent or excipient must be compatible with the other ingredients of the formulation and not deleterious to the recipient thereof.

The lipids include 1,2-dimyristoyl-sn-glycero-3-phosphocholine (DMPC), 1,2-dimyristoyl-sn-glycero-3-phosphorylglycerol (DMPG), DMPC/DMPG, lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, or combinations thereof.

The lipids of Formula (I):

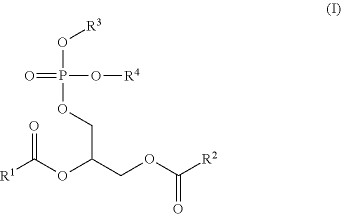

wherein, R¹ is a C₁-C₂₀ branched or unbranched hydrocarbon possessing 0-10 double bonds, 0-10 triple bonds or a combination of 0-10 double and triple bonds; R² is a C₁-C₂₀ branched or unbranched hydrocarbon possessing 0-10 double bonds, 0-10 triple bonds or a combination of 0-10 double and triple bonds;
R³ is

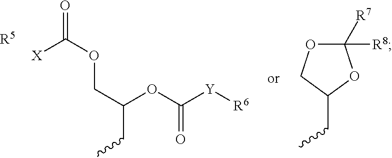

R⁴ is H or a pharmaceutically acceptable cation, wherein incorporation of said pharmaceutically acceptable cation results in a salt; R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I; R⁷ is a C₀-C₂₀ branched or unbranched hydrocarbon possessing 0-10 double bonds, 0-10 triple bonds or a combination of 0-10 double and triple bonds; R⁸ is H or a C₀-C₂₀ branched or unbranched hydrocarbon possessing 0-10 double bonds, 0-10 triple bonds or a combination of 0-10 double and triple bonds; X is a direct linkage, CH₂, O or NH; Y is a direct linkage, CH₂, O or NH; and, each stereogenic center is independently R, S or racemic. The lipids are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the lipid may be formulated to provide, e.g., maximum and/or consistent dosing for intrapleural administration. While the lipid may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

The lipid may be administered in the form of a liposome, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

Methods and analysis. A 3+3 expanded cohort for predefined dose escalation levels or until a predefined number of dose-limiting toxicities are reached. Participants will be administered a single dose of liposomal curcumin (LipoCurc™, SignPath Pharma, Inc.) via their existing TIPC as a sequential enrolling case series with the following dose cohorts: 100, 200 and 300 mg/m², but cam also be administered at 50, 100, 150, 200, 250, 300 or 350 mg/m². Primary endpoints are determination of the maximum tolerated dose within the predetermined dose range, and determination of the feasibility of intrapleural administration of liposomal curcumin via an existing TIPC. Secondary endpoints include determination of the safety and tolerability of intrapleural administration of liposomal curcumin, median overall survival, effects on quality of life and on feelings of breathlessness, and the pharmacokinetics and concentrations of curcumin from the plasma and the pleural fluid. Important inclusion criteria include age >18 years, an existing TIPC, a pleural biopsy or pleural fluid cytology proven diagnosis of malignant pleural effusion and for whom no anti-tumor therapy of proven benefit is available or has been previously declined, eastern co-operative group performance status <2. Ethics and dissemination: The study protocol has been approved by the Southern Adelaide Local Health Network HREC (approval number: HREC/20/SAC/11). Study results will be published in peer-reviewed journals, and presented at conferences, in field of medical oncology and respiratory medicine. Trial registry and registration number: Australian and New Zealand Clinical trial registry, ACTRJN12620001216909.

This is the first study in which curcumin of any formulation will be delivered directly into the pleural cavity in humans. Due to the pharmacokinetic nature of curcumin, namely poor bioavailability and rapid degradation, direct intrapleural delivery of liposome encapsulated curcumin may better provide clinically relevant concentrations of curcumin to tumours at this site. The study design does not allow for the evaluation of the safety of multiple doses of liposomal curcumin. Multiple doses can be provided for sustained symptomatic relief, or a potential antineoplastic effect.

Malignant pleural effusion. A malignant pleural effusion (MPE) is the accumulation of fluid in the pleural cavity as a result of primary or secondary pleural malignancy. The most prevalent causes of MPE are late stage breast and lung cancers that have metastasized to the pleural or mediastinal lymph nodes.¹ Patients presenting with an MPE have a poor prognosis, with median survival times between 1 and 12 months depending of the type and stage of the underlying malignancy.²⁻⁴ Controlling the pleural effusion is an integral part of managing patient symptoms as a MPE causes significant discomfort, breathing difficulties and commonly recurs after the initial therapeutic drainage.¹ Recurrent pleural effusion can be managed by either talc pleurodesis or insertion of a tunneled indwelling pleural catheter (TIPC).⁵⁻⁷ At present, there is a lack of evidence supporting the use of anti-cancer therapies as an alternative to MPE drainage⁶, and so the pursuit of new possible treatments, or adjuvant therapy options and approaches for MPE, from early-intervention all the way through to palliative stages, remains an open challenge.

Curcumin and cancer. Curcumin is a polyphenol derived from the spice turmeric, which can modulate numerous pathways involved in carcinogenesis, including those controlling inflammation, cell cycle progression, angiogenesis and cell survival. Curcumin also has the potential to help to reduce pleural fluid production since it can reduce numerous factors involved in fluid accumulation including vascular endothelial growth factor-A, interlukin-6, signal transducer and activator of transcription 3, sphingosine phosphate, indolamine 2,3 dioxygenase and tumor necrosis factor-alpha.^{1 8-10} The translation of these anti-cancer effects into a clinical setting has been hampered because of curcumin's low solubility, instability at physiological pH, low bioavailability and rapid molecular transformation and degradation in the blood.¹¹ In order to combat this, a variety of curcumin formulations and drug delivery systems have been developed to improve its stability, solubility and bioavailability in vivo including lipid-based vehicles such as curcumin-loaded micelles and liposomes.¹²

Liposomal curcumin. Liposomes are phospholipid vesicles that are used as delivery systems for drugs to reduce early degradation and improve stability, biodistribution and cellular uptake.^{13 14} A pharmaceutical-grade, liposome-encapsulated synthetic curcumin, called ‘LipoCurc™’ is currently the only liposomal curcumin formulation that has been administered intravenously in humans in both healthy humans and advanced cancer patients. In cancer patients, liposomal curcumin (100-300 mg/m²) was administered intravenously over a 6-8 h period, weekly for eight weeks or until the disease progressed or intolerable toxicity was observed. A significant tumor marker response and temporary clinical benefit were observed in two patients after receiving 300 mg/m² of liposomal curcumin.¹⁵ However, following intravenous drug delivery, a drug may be unevenly redistributed to organs tissues, and fluids¹⁶ and thus only a small proportion of the administered dose may reach the pleural cavity.

Rationale for phase 1 trial of intrapleural administration of liposomal curcumin. The position of the tumor cells adjacent to the pleural cavity provides a unique opportunity to administer therapeutics directly to the tumor site. Intrapleural liposomal curcumin therapy offers several potential advantages over intravenous therapy in patients with primary and secondary malignancies of the pleura. Most importantly, it allows the delivery of a high dose of curcumin directly to the site of the tumor and, in reality; this may be the only method to deliver clinically relevant concentrations of curcumin to the pleural cavity whilst minimizing potential systemic toxicity. Liposome encapsulated chemotherapeutic drugs have been administered into the pleural cavity in both phase I and phase II clinical trials in humans with malignant pleural mesothelioma before to some effect.^{17 18} Therapeutic delivery via an existing TIPC means patients could be given tumor-site targeted therapies while also avoiding any additional instrumentation of the pleura, therefore reducing procedural risk.

Justification of intrapleural curcumin dose and escalation schedule. The safety level of three pre-determined single escalating dose levels of liposomal curcumin (LipoCurc™) is determined. Dosage levels will be body surface area adjusted to 100 mg/m², 200 mg/m² and 300 mg/m². These dose concentrations are below the previously established safe systemic dosage levels of liposomal curcumin determined in healthy adults (maximum tolerated dose (MTD) 400 mg/m²)¹⁹, and in line with intravenous dosage levels assessing liposomal curcumin efficacy for the treatment of metastatic cancers in phase II clinical trials.¹⁵ Body surface area adjusted doses have been used in clinical trials that assessed the safety and efficacy of drugs administered into the pleural and peritoneal cavities in humans, including liposome-entrapped antineoplastic therapies. 17 18 20 21

Pre-clinical studies in healthy rats also demonstrated low systemic curcumin absorption following intrapleural administration of liposomal curcumin (LipoCurc™). Furthermore, the rat study showed that intrapleural doses of liposomal curcumin equivalent to 300 mg/m² in humans did not cause any pleural pathology.²²

By contrast, the MTD for liposome-entrapped chemotherapy delivered to the pleural cavity in humans was 50% higher than the MTD after intravenous administration of the same dose.¹⁸ In light of these studies, it is not anticipated that the MTD will be reached at the highest planned dosage level. Alternatively, the liposomal curcumin or liposomal curcuminoids are administered at a dose of 125, 150, 200, 250, 300, 325, 350, 400, 450, 500, or 600 mg/m²; or the liposomal curcumin or liposomal curcuminoids are administered at a dose of 100 to 600 mg/m², over 2 to 6 hours; or the liposomal curcumin or liposomal curcuminoids are administered at a dose of greater than 300 to 600 mg/m², over 2 to 4 hours; or the liposomal curcumin or liposomal curcuminoids are administered at a dose of greater than 300 to 600 mg/m², over 2 or less; or the liposomal curcumin or liposomal curcuminoids are administered for 2, 3, 4, 5, 6, or 7 hours; or the liposomal curcumin or liposomal curcuminoids are administered at a dose of greater than 300 mg/m² over 2, 3, 4, 5, 6, 7, or 8 hours.

The Standard Protocol Items: Recommendations for Interventional Trials (SPIRIT) checklist was used when preparing this protocol²³. The safety and feasibility of a single dose of intrapleural liposomal curcumin (LipoCurc™) in patients with malignant pleural effusion is determined by administering the liposomal curcumin via their existing TIPC. The following are determined: Primary objectives: (1) If the MTD of intrapleural liposomal curcumin is reached within the pre-determined escalating dose range in people with an advanced MPE; (2) The feasibility of intrapleural administration of liposomal curcumin via an existing TIPC.

Secondary objectives: (1) To evaluate safety and tolerability by determining: the rates of toxicities based on the National Cancer Institute Common Terminology Criteria for Adverse Events v5.0 (NCI CTCAE v5.0); the effects of the study intervention on quality of life based on the average scores as assessed by the European Organisation for Research and Treatment of Cancer Quality of life Questionnaire© (EORTC-QLQ©)²⁴; the effects of the study intervention on feeling of breathlessness, based on the average scores as assessed by the Visual Analogue Scale for Breathlessness survey (VASB survey); and/or Median overall survival. (2) To evaluate the pharmacokinetics of curcumin in all participants following administration of a single dose of liposomal curcumin in to the intrapleural cavity by measuring the concentrations of curcumin and its metabolites in the plasma and, where possible, pleural fluid. (3) To evaluate any evidence of anti-tumor activity as indicated by clinical outcomes, in conjunction with cellular and molecular analysis of participant's biospecimens collected for diagnostic purposes and surplus to diagnostic needs.

Study design. Open label, single-center, phase 1 trial modelled on a standard 3+3 dose escalation design.

Subject Population. Target population. People in palliative care with a cyto-pathologically or histo-pathologically proven diagnosis of MPE, and where the insertion of a TIPC is indicated by the treating clinician.

Inclusion criteria: Age ≥18 years; an existing diagnosis of malignant pleural effusion proven by either: pleural biopsy, or pleural fluid cytology in conjunction with typical radiological and clinical findings; individuals who have: failed to respond to approved systemic therapies (chemotherapy, immune therapy or molecular targeted therapies), or who have progressive cancers following initial response to these therapies, and for whom no anti-tumor therapy of proven benefit is available at study enrolment or who have declined systemic therapies or are deemed not suitable for systemic therapies after consultation with a medical oncologist; recurrent symptomatic pleural effusion where insertion of a TIPC is clinically indicated; An Eastern Co-operative Group Performance Status of 0-2; and able to give signed informed consent.

Exclusion criteria: Any comorbidities or conditions that the investigator considers the patient should not participate in the study include: evidence of active hepatitis, people with a diagnosis of lymphoma or hematological cancer, people with a history of hemolytic anemia, people with unresolved toxicities from prior systemic anti-cancer therapy, or people with an unstable cardiac condition as determined by the study investigators; women who are pregnant and/or breastfeeding, and/or of childbearing age not taking contraceptive measures to avoid pregnancy; people with mental impairment, or an unstable medical condition other than cancer that may interfere with their ability to provide informed consent or ability to cooperate and participate in the study; people whose primary language is not English; people who are taking anticoagulation medication, including warfarin, clexane and/or direct oral anticoagulants.

Participant screening and registration. All inclusion and exclusion criteria are assessed as a part of standard practice of care and/or before TIPC insertion. With no exceptions, prospective participants that meet all the inclusion criteria, and none of the exclusion criteria, will be eligible for participation and will be offered the opportunity to take part in the clinical trial. Written informed consent must be signed and dated by the participant, and the investigator, prior to any study-specific procedures or activities.

Participant enrolment and treatment plan. Participants enter the trial and are treated as a consecutively enrolling case series as outlined in Table 1.

TABLE 1
Dosing of intrapleural Lipocurc ™ based on 3 + 3 design.

| | | Lipocurc ™ | |
|---|---|---|---|
| | No of | dose given | |
| Cohort | patients | on day 1 | Escalation |
| 1 | 3 | 100 mg/m² | If no dose-limiting toxicity is seen |
| | | | after a minimum of 3 patients have |
| | | | completed treatment in cohort 1, |
| | | | enrollment will commence for cohort |
| | | | 2 participants. |
| 2 | 3 | 200 mg/m² | If no dose-limiting toxicity is seen |
| | | | after a minimum of 3 patients have |
| | | | completed treatment in cohort 2, |
| | | | enrollment will commence for cohort |
| | | | 3 participants. |
| 3 | 6 | 300 mg/m² | If no dose-limiting toxicity is seen |
| | | | after three patients have completed |
| | | | treatment in cohort 3, there will be |
| | | | no further dose escalation. This cohort |
| | | | will be expanded to six patients |
| | | | if maximum tolerated dose has not |
| | | | been reached. |

Insertion of TIPC. The insertion of a TIPC occurs at least one week before the administration of the liposomal curcumin, to allow for monitoring and treatment of any potential post-procedure complications. Patients will remain in the hospital for 24 to 48 hours after TIPC insertion (as per our local protocol) and medical/nursing staff will monitor patients' until deemed medically fit to be discharged by the clinician. Pleural fluid drainage is performed via a vacuum bottle system (PleurX™ Drainage System). The volume and frequency of drainage is determined and managed by the clinical team. Drained pleural fluid obtained from the effusion is collected, and cells separated by centrifugation, for pathology monitoring and testing. Baseline studies is performed after TIPC insertion, as outlined in Table 2. Participants' renal function is estimated by Estimated Glomerular Filtration Rate (eGFR) using their body weight and urea and electrolytes (UEC) prior to the computed tomography (CT) scan to determine if a CT with intravenous contrast can be performed. Participants with an eGFR >30 mL/min/1.73 m² may have intravenous contrast in accordance with the 2011 Iodinated Contrast Guidelines from the Royal Australia and New Zealand College of Radiologists. Participants with an eGFR <30 mL/min/1.73 m² will not have intravenous contrast for CT scans.

TABLE 2
Scheduled visits and associated procedures required during the
phase 1 study assessing the safety and feasibility of intrapleural
administration of Lipocurc ™ via an existing TIPC.

| | | Blood | | Pleural | | | | |
|---|---|---|---|---|---|---|---|---|
| | | tests -incl. | | effusion | | EORTC | | |
| | | Circulating | | fluid | VASB | QLQ- | Chest | Chest |
| Week | Location | Curcumin | Vitals | collection | survey | C30 | X-ray | CT |
| Week −1 | In- | ✓* | ✓* | ✓* | ✓ | ✓ | ✓* | x |
| TIPC | patient | | | | | | Post- | |
| insertion* | 24-48 h* | | | | | | TIPC | |
| Week 0 | Clinic + | ✓ | ✓ | ✓ | ✓ | ✓ | ✓ | ✓ |
| IPA- | In- | Pre-IPA, + | | Pre-IPA | 2, 24, | 2, 24, | 48 h | Pre- |
| Lipocurc ™ | patient | 2, 24, & | | | 48 h | 48 h | Post- | IPA |
| | 48 h | 48 h | | | Post- | Post- | IPA | |
| | | Post-IPA | | | IPA | IPA | | |
| Week 1 | Clinic | ✓ | ✓ | If | ✓ | ✓ | ✓ | x |
| | | | | possible | | | | |
| Week 4 | Clinic* | ✓ | ✓* | If | ✓ | ✓ | ✓ | x |
| | | | | possible | | | | |
| Week 8 | Clinic | ✓ | ✓ | If | ✓ | ✓ | ✓ | ✓ |
| | | | | possible | | | | |
| Week 12 | Clinic | ✓ | ✓ | If | ✓ | ✓ | ✓ | x |
| | | | | possible | | | | |
| Week 24 | Clinic | ✓ | ✓ | If | ✓ | ✓ | ✓ | x |
| | | | | possible | | | | |

*Activities and procedures required for routine standard of care for TIPC insertion and clinical care. TIPC, Tunnelled Indwelling Catheter; IPA-Lipocurc ™, Intrapleural administration of Lipocurc ™; Blood tests include, FBC, UEC, LFT, CRP; Vitals monitoring includes, blood pressure, pulse, Oxygen (O2) saturation; VASB, Visual Analogue Scale for Breathlessness; EORTC QLQ-C30, European Organisation for Research and Treatment of Cancer Quality of Life Questionaire-C30; CT, Computed Tomogram.

Intrapleural administration of liposomal curcumin via an existing TIPC. A week after the insertion of the TIPC, participants attend the clinic as an outpatient for suture removal and vacuum bottle drainage. Baseline studies will be taken, as outlined in Table 2. Participants are then admitted as a hospital in-patient for the intrapleural liposomal curcumin administration. Before intrapleural administration of the liposomal curcumin, pleural fluid is drained via the TIPC using the vacuum drainage bottle system. Liposomal curcumin is prepared under aseptic conditions by the treating medical team and administered at room temperature through the TIPC via an adaptor port (PleurX™ catheter Access Kit). Drug delivery will take 10 to 15 minutes and is followed by sequential 10 mL flushes of room temperature sterile 0.9% saline until the TIPC is visibly clear of the yellow-colored liposomal curcumin solution. Participants are admitted as an in-patient for 48 hours to allow time to monitor for any potential serious adverse events. The participant is monitored by medical and nursing staff, with observations of blood pressure, pulse, oxygen saturation and temperature every 15 minutes for 1 hour, and then every hour for 4 hours, then at 4 hourly intervals up to 48 hours in total. Additional post-procedural assessments is performed according to the schedule in Table 2.

Any adverse reactions are evaluated by the National Cancer Institute Common Toxicity Evaluation Criteria (NCI CTC) and treated appropriately at the discretion of the respiration physician. Upon discharge, participants are referred for at home drainage of the effusion.

A minimum period of at least one week between administration of liposomal curcumin and consecutively enrolling participants is instituted to check for toxicities or serious adverse events. This timeframe is in line with normal clinical practices for monitoring post-intrapleural treatments for pleural effusion. Furthermore, in pre-clinical studies the inventors did not detect any curcumin in the plasma of rats one week after intrapleural administration of liposomal curcumin (dose equivalent to 300 mg/m²).²² Escalation to the next dosage level will be stopped if one third or greater (≥33%) of cohort participants experience dose limiting toxicities. If the MTD level is reached, another three patients will be accrued at the previous dose level to assure tolerability in subsequent phase clinical trials.

Follow up post single dose of intrapleural liposomal curcumin. One week after the intrapleural administration of the single dose of liposomal curcumin, participants will be assessed as an outpatient at the Respiratory Clinic at Flinders Medical Centre, and monitored as outlined in Table 2. Outpatient monitoring is repeated at 4, 8, 12 and 24 weeks post-intrapleural liposomal curcumin administration. Where possible, any pleural fluid drained at follow-up clinic visits is collected for analysis.

Concomitant medication and reporting. Prohibited medications, and subject to exclusion from study participation: Anticoagulation medication; including Warfarin, Clexane and/or Direct Oral Anticoagulants. There are no diet restrictions on trial participants during the study. Numerous studies have shown that orally consumed dietary curcumin—in food or as a supplement—is poorly absorbed beyond the gut mucosa, with either undetectable or negligible levels of curcumin measured in blood serum or urine.²⁵⁻²⁷ Most ingested curcumin is excreted, unchanged, in the faeces.^{28 29} Concomitant medications will not be recorded during the study.

Outcomes Endpoints and Other Measures. Participants will be assessed for up to a maximum of 24 weeks post administration of the liposomal curcumin. In the event a participant dies prior to completing study participation, date of death will be recorded, and data collected prior to death will be included in study analyses.

Outcomes will be measured at weeks 0, 4, 8, 12 and 24 post-administration of liposomal curcumin for:

Maximum Tolerated Dose (MTD). Maximum tolerated dose (MTD) is defined as the highest dose level at which ≤33% of participants experience dose-liming toxicity (DLT).³⁰ DLTs will be assessed according to the Common Toxicity Evaluation Criteria according to the National Cancer Institute version 5 (NCI-CTCAE v5.0, 2017) guidelines.³¹ DLTs are defined as the following adverse events if they develop within one week of receiving the trial intervention and with a reasonable suspected causal relationship to intrapleural Lipocurc™.

DLTs include:

Hemolysis. NCI-CTC Grade 2 evidence of hemolysis and ≥2 gram decrease in hemoglobin, confirmation of a causal relationship to the study medication according to the investigator.

Non-Haematological Toxicity. (1) Any NCI-CTC grade 3 or higher non-haematological toxicity, excluding nausea and vomiting which responds to antiemetic treatment, and alopecia. (2) Neuro-cerebellar: Intention tremor, slurred speech, nystagmus, dysmetria

Other Haematological Toxicity. (1) NCI-CTC Grade 3 anaemia, (2) NCI-CTC Grade 4 platelet toxicities, (3) NCI-CTC Grade 4 granulocyte toxicity ≥7 days, (4) Febrile neutropenia: defined as an absolute neutrophils count <500/mm³ and fever either as 2 elevations of oral temperature >38° C. with one hour interval or a single oral temperature >38.5° C., provided that this single episode is not clearly related to other events.

Adverse events (AEs) (worst grade according to the NCI-CTCAE v5.0). Any adverse reaction events are evaluated by the NCI-CTCAE v5.0 guidelines to classify and grade the intensity of AEs during trial participation. See Safety Reporting for the definition of an AE and reporting of SAEs.

Effects of treatment intervention on quality of life. Based on average scores provided from participant responses to VASB survey (supplementary FIG. 1) and EORTC QLQ-C30© questionnaire.

Overall survival. Overall survival is defined as the interval from on-study enrolment date, until the date of death from any cause, or cessation of study participation (the date of last known study follow-up visit alive and/or final study follow up visit), whichever comes first.

Safety Reporting. Definitions. An adverse event (AE) is any medical occurrence in a trial participant administered a pharmaceutical product and which does not necessarily have a causal relationship with this treatment.

AEs include: an exacerbation, or an unexpected increase in the intensity or frequency of a pre-existing condition (other than the malignant pleural effusion condition under investigation), including intermittent or episodic conditions; significant or unexpected worsening or exacerbation of the malignant pleural effusion; a suspected drug interaction; an intercurrent illness; any clinically significant laboratory abnormality that requires clinical intervention or further investigation (beyond ordering a laboratory test), injury or accidents.

An AE does not include: anticipated day-to-day fluctuations of any pre-existing conditions, including the malignant pleural effusion and/or underlying malignancy. Signs and symptoms of the disease under study that do not represent a significant worsening or exacerbation. Expected progression of the malignant pleural effusion or underlying malignancy. AEs with severity Grades 1 or 2 as defined in the NCI-CTCAE v5.0 guidelines, must be reported to the principal investigator and recorded.

Serious AEs include: any untoward medical occurrence which fulfils the definition of an AE (see above) and additionally is: fatal; life threatening; results in hospitalisation or prolongs hospital stay; disabling/incapacitating; a congenital abnormality; Any important medical occurrence which the investigator regards as serious based on appropriate medical judgment.

Laboratory abnormalities identified as critical to safety evaluations that also require immediate reporting include; low red blood cell count, or other indicators of haemolytic disorders.

Serious AEs (SAEs) with severity of Grades 3, 4 or 5, must be immediately reported to the principal investigator within 1 working day of the investigator becoming aware of the event and recorded. The principal investigator must notify the study sponsor and the local Human Research Ethics Committee as required.

Investigators must continue to monitor all participants with SAEs until the event has subsided, stabilised, otherwise explained, or the participant is lost to follow up.

All AEs, including Adverse Drug Reactions (ADRs), SAEs, or Serious Adverse Drug Reactions (serious ADR), will be prospectively tabulated for the duration of the trial to inform ongoing safety and toxicity monitoring by the Safety Review/Dose Escalation Committee, annual reports to the SAC HREC, and the final study report.

Pregnancy. The effects of liposomal curcumin on the unborn child and on the newborn baby are not known. Both male and female participants will be strongly advised to use effective contraception for the period of time they are actively participating in this research project, and for a period of 90 days after receiving the single dose of the test drug. As a precaution, in the event a participant becomes pregnant during study participation, they will be withdrawn from the study. Male participants who father a child while participating in the research project and/or up to 90 days after administration of the liposomal curcumin should be reported to the study investigators. The study doctor will advise on any medical attention required for the pregnant partner should this be necessary.

Statistical Considerations and Data Analysis Plan. This is a Phase 1 uncontrolled, open label, dose escalation study primarily for the assessment of safety using a standard 3+3 dose escalation design,³⁰ therefore does not require sample size justification. The dose escalation will be continued until the predefined dose level and/or the MTD is reached. Analyses will be primarily descriptive and observational to characterize DLTs, and response rates.

Test Drug Information—LipoCurc™. The liposomal curcumin test drug, LipoCurc™ was manufactured by SignPath Pharma, Inc. (US), and tested and packaged and labelled by Polymun Scientific, Austria in compliance with Good Manufacturing Practice (GMP). Curcumin is synthesised at under GMP conditions to 99.2% purity. The use of LipoCurc™ for this phase 1 trial subject to conditional on its registration with the Therapeutic Goods Administration of Australia by the study sponsor, Flinders University, under the Clinical Trial Notification scheme.

Study Administration. Ethics and dissemination. This single site study was approved by the Southern Adelaide Local Health Network Human Research Ethics Committee (approval number: HREC/20/SAC/11). On study completion, a manuscript will be prepared for publication. This study will be conducted in accordance with the Integrated Addendum to ICH E6(R1): Guideline For Good Clinical Practice ICH E6(R2) (© International Council For Harmonisation Of Technical Requirements For Pharmaceuticals For Human Use (ICH), 2016), the Note for Guidance on Good Clinical Practice (CPMP/ICH/135/95) annotated with TGA comments (Therapeutic Goods Administration DSEB July 2000), and in compliance with applicable laws and regulations. The study is performed in accordance with the Australian National Statement on Ethical Conduct in Human Research (updated 2018, © Commonwealth of Australia 2007), and the NHMRC Australian Code for Responsible Conduct of Research (© Commonwealth of Australia 2007), and the principals laid down in the Declaration of Helsinki 2008 by the World Medical Assembly.

Recruitment of Participants. Prospective participants are recruited from the pool of patients presenting at Flinders Medical Centre, South Australia, for the management of their malignant pleural effusion. Whenever possible, an independent respiratory clinician working at FMC will directly approach and discuss possible recruitment to the study with pre-identified (pre-screened) candidates for study participation at the time that standard TIPC insertion is explained to patients. If they express and interest in the study, they are provided with a copy of the study participant information and consent form by a member of the research team inviting them to voluntarily participate in the research study. Prospective participants are encouraged to discuss trial participation with others, including with their family and their own doctor, as well as with the study team prior to granting their consent. Prospective participants are about 1 to 2 weeks to consider if they will grant their consent. Persons who decide to participate meet with a study investigator to sign a consent form.

Consent. Pre-screening of prospective participants is conducted in compliance with the waiver of consent granted for this process. Each participant's voluntary decision to take part in the study is established by written informed consent documented on the participant information and consent form which is signed and dated by the participant and by the witnessing consenting clinician, or member of the trial research team. All participants are provided with a signed and dated copy of their informed consent. Written informed consent must be obtained prior to any study specific procedures or activities.

Confidentiality. All data generated in this study will remain confidential in accordance with applicable privacy acts and regulations. All data are securely stored and made only available to members of the study research team and who have signed a confidentiality agreement. Study data is analyzed and reported in a largely aggregated manner, and trial participants are anonymous in any presentations or publications arising from this study.

Protocol amendments. Any changes and amendments to the protocol can only be made by the principal investigator. Protocol changes can only be implemented upon approval by the institutional Human Research Ethics Committee (HREC).

Data handling and record keeping. Trial data required for the monitoring and analysis of the study is recorded on the case reports forms and associated documentation of laboratory investigations and outcomes. Accuracy of the completed case report forms is indicated by the signing by the study investigator. All study data are maintained for a minimum of 15 years following completion of the study in accordance with the Australian Code for the Responsible Conduct of Research³².

Study monitoring. Safety review Dose-escalation Committee. A Safety Review/Dose Escalation Committee will function in consultation with independent respiratory physicians to review emerging safety and pharmacokinetic data and to make key decisions about the trial. In particular, the Safety Review/Dose Escalation Committee, will consult on advances in participant recruitment, when appropriate to advance dose escalation, and when to terminate the study.

Audit and inspection. Monitoring will include centralized review of data collection and other study documents for protocol compliance, data accuracy and completeness. All study documents are made available to the study Sponsor, and representatives of regulatory bodies for monitoring or audit purposes.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Psallidas I, Kalomenidis I, Porcel J M, et al. Malignant pleural effusion: from bench to bedside. Eur Respir Rev 2016; 25(140):189-98. doi: 10.1183/16000617.0019-2016
2. Zamboni M M, da Silva C T, Jr., Baretta R, et al. Important prognostic factors for survival in patients with malignant pleural effusion. BMC Pulm Med 2015; 15:29. doi: 10.1186/s12890-015-0025-z
3. Porcel J M, Gasol A, Bielsa S, et al. Clinical features and survival of lung cancer patients with pleural effusions. Respirology 2015; 20(4):654-9. doi: 10.1111/resp.12496
4. Clive A O, Kahan B C, Hooper C E, et al. Predicting survival in malignant pleural effusion: development and validation of the LENT prognostic score. Thorax 2014; 69(12):1098-104. doi: 10.1136/thoraxjnl-2014-205285
5. Davies H E, Mishra E K, Kahan B C, et al. Effect of an indwelling pleural catheter vs chest tube and talc pleurodesis for relieving dyspnea in patients with malignant pleural effusion: the TIME2 randomized controlled trial. JAMA 2012; 307(22):2383-9. doi: 10.1001/jama.2012.5535
6. Bibby A C, Dorn P, Psallidas I, et al. ERS/EACTS statement on the management of malignant pleural effusions. Eur J Cardiothorac Surg 2019; 55(1):116-32. doi: 10.1093/ejcts/ezy258
7. Feller-Kopman D J, Reddy C B, DeCamp M M, et al. Management of Malignant Pleural Effusions. An Official ATS/STS/STR Clinical Practice Guideline. Am J Respir Crit Care Med 2018; 198(7):839-49. doi: 10.1164/rccm.201807-1415ST
8. Yeh H H, Lai W W, Chen H H, et al. Autocrine IL-6-induced Stat3 activation contributes to the pathogenesis of lung adenocarcinoma and malignant pleural effusion. Oncogene 2006; 25(31):4300-9. doi: 10.1038/sj.onc.1209464
9. Stathopoulos G T, Kollintza A, Moschos C, et al. Tumor necrosis factor-alpha promotes malignant pleural effusion. Cancer Res 2007; 67(20):9825-34. doi: 10.1158/0008-5472.CAN-07-1064
10. Shanmugam M K, Rane G, Kanchi M M, et al. The multifaceted role of curcumin in cancer prevention and treatment. Molecules 2015; 20(2):2728-69. doi: 10.3390/molecules20022728
11. Heger M, van Golen R F, Broekgaarden M, et al. The molecular basis for the pharmacokinetics and pharmacodynamics of curcumin and its metabolites in relation to cancer. Pharmacol Rev 2014; 66(1):222-307. doi: 10.1124/pr.110.004044
12. Liu L, Sun L, Wu Q, et al. Curcumin loaded polymeric micelles inhibit breast tumor growth and spontaneous pulmonary metastasis. Int J Pharm 2013; 443(1-2):175-82. doi: 10.1016/j.ijpharm.2012.12.032
13. Feng T, Wei Y, Lee R J, et al. Liposomal curcumin and its application in cancer. Int J Nanomedicine 2017; 12:6027-44. doi: 10.2147/IJN.S132434
14. Sercombe L, Veerati T, Moheimani F, et al. Advances and challenges of liposome assisted drug delivery. Front Pharmacol 2015; 6:286. doi: 10.3389/fphar.2015.00286
15. Greil R, Greil-Ressler S, Weiss L, et al. A phase 1 dose-escalation study on the safety, tolerability and activity of liposomal curcumin (Lipocurc™) in patients with locally advanced or metastatic cancer. Cancer Chemother Pharmacol 2018; 82(4):695-706. doi: 10.1007/s00280-018-3654-0
16. Matabudul D, Pucaj K, Bolger G, et al. Tissue distribution of (Lipocurc) liposomal curcumin and tetrahydrocurcumin following two- and eight-hour infusions in Beagle dogs. Anticancer Res 2012; 32(10):4359-64.
17. Lu C, Perez-Soler R, Piperdi B, et al. Phase II study of a liposome-entrapped cisplatin analog (L-NDDP) administered intrapleurally and pathologic response rates in patients with malignant pleural mesothelioma. J Clin Oncol 2005; 23(15):3495-501. doi: 10.1200/JCO.2005.00.802
18. Perez-Soler R, Shin D M, Siddik Z H, et al. Phase I clinical and pharmacological study of liposome-entrapped NDDP administered intrapleurally in patients with malignant pleural effusions. Clin Cancer Res 1997; 3(3):373-9.
19. Storka A, Veelar B, Klickovic U, et al. Safety, tolerability and pharmacokinetics of liposomal curcumin in healthy humans. Int J Clin Pharmacol Ther 2015; 53(1):54-65. doi: 10.5414/CP202076
20. Vatandoust S, Bright T, Roy A C, et al. Phase I open-label trial of intraperitoneal paclitaxel in combination with intravenous cisplatin and oral capecitabine in patients with advanced gastric cancer and peritoneal metastases (IPGP study): study protocol. BMJ Open 2019; 9(5):e026732. doi: 10.1136/bmjopen-2018-026732
21. Delgado G, Potkul R K, Treat J A, et al. A phase I/II study of intraperitoneally administered doxorubicin entrapped in cardiolipin liposomes in patients with ovarian cancer. Am J Obstet Gynecol 1989; 160(4):812-7; discussion 17-9. doi: 10.1016/0002-9378(89)90296-2 [published Online First: 1989/04/01]
22. Hocking A, Tommasi S, Sordillo P, et al. The Safety and Exploration of the Pharmacokinetics of Intrapleural Liposomal Curcumin. Int J Nanomedicine 2020; 15:943-52. doi: 10.2147/IJN.5237536
23. Chan A-W, Tetzlaff J M, Gotzsche P C, et al. SPIRIT 2013 explanation and elaboration: guidance for protocols of clinical trials. BMJ: British Medical Journal 2013; 346:e7586. doi: 10.1136/bmj.e7586
24. Aaronson N K, Ahmedzai S, Bergman B, et al. The European Organization for Research and Treatment of Cancer QLQ-C₃₀: a quality-of-life instrument for use in international clinical trials in oncology. J Natl Cancer Inst 1993; 85(5):365-76. doi: 10.1093/jnci/85.5.365 [published Online First: 1993/03/03]
25. Cheng A L, Hsu C H, Lin J K, et al. Phase I clinical trial of curcumin, a chemopreventive agent, in patients with high-risk or pre-malignant lesions. Anticancer Res 2001; 21(4B):2895-900.
26. Sharma R A, Euden S A, Platton S L, et al. Phase I clinical trial of oral curcumin: biomarkers of systemic activity and compliance. Clin Cancer Res 2004; 10(20):6847-54. doi: 10.1158/1078-0432.CCR-04-0744
27. Carroll R E, Benya R V, Turgeon D K, et al. Phase IIa clinical trial of curcumin for the prevention of colorectal neoplasia. Cancer Prev Res (Phila) 2011; 4(3):354-64. doi: 10.1158/1940-6207.CAPR-10-0098
28. Ravindranath V, Chandrasekhara N. Absorption and tissue distribution of curcumin in rats. Toxicology 1980; 16(3):259-65.
29. Wahlstrom B, Blennow G. A study on the fate of curcumin in the rat. Acta Pharmacol Toxicol (Copenh) 1978; 43(2):86-92.
30. Le Tourneau C, Lee J J, Siu L L. Dose escalation methods in phase I cancer clinical trials. Journal of the National Cancer Institute 2009; 101(10):708-20. doi: 10.1093/jnci/djp079 [published Online First: 05/12]
31. Common Terminology Criteria for Adverse Events (CTCAE) v5.0. published online on The National Cancer Institute website at https://ctep.cancer.gov/protocolDevelopment/electronic_applications/ctc.htm #ctc_50, 2018.
32. Australian Code for the Responsible Conduct of Research Camberra: National Health and Medical Research Council, 2018.

1. A method for treating malignant pleural effusion in a human patient comprising:
administering to the human patient a therapeutically effective amount of a liposomal curcumin or liposomal curcuminoids intrapleurally, wherein the therapeutically effective amount is sufficient to reduce or treat the malignant pleural effusion.

2. The method of claim 1, wherein a pleural space or cavity is adjacent to at least one of the lung, heart, kidney, or liver.

3. (canceled)

4. (canceled)

5. The method of claim 1, wherein the liposomal curcumin or liposomal curcuminoids are administered at a dose of 50, 100, 125, 150, 200, 250, 300, 325, 350, 400, 450, 500, or 600 mg/m², over 2 hours, 2 to 4 hours, 2 to 6 hours, or administered for 2, 3, 4, 5, 6, or 7 hours; or
the dose is 300, 325, 350, 400, 450, 500, or 600 mg/m², over 2 hours, 2 to 4 hours, 2 to 6 hours, or administered for 2, 3, 4, 5, 6, or 7 hours; or
the dose greater than 350 mg/m² over 2, 3, 4, 5, 6, 7, or 8 hours.

6. (canceled)

7. (canceled)

8. (canceled)

9. (canceled)

10. The method of claim 1, wherein the curcumin or curcuminoids are chemically synthesized curcumin or curcuminoids.

11. The method of claim 1, wherein the liposome comprises at least one of: 1,2-dimyristoyl-sn-glycero-3-phosphocholine (DMPC), 1,2-dimyristoyl-sn-glycero-3-phosphorylglycerol (DMPG), DMPC/DMPG, lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, or combinations thereof.

12. The method of claim 1, wherein the liposomal curcumin or curcuminoids are curcumin/curcuminoid:liposome complex, wherein the curcumin comprises between 2 to 9 weight percent of the curcumin/curcuminoid:liposome complex, wherein the curcumin is at least one of natural or synthetic curcumin and wherein the curcumin/curcuminoid:liposome complex has a ratio of curcumin to lipid (weight to weight) of 1:7.5 to 1:10, wherein the lipid combination is selected from: DMPC; DMPC:Chol 9:1; DMPC:DMPG 9:1; DMPC:Chol:DMPG 8:1:1;
DPPC:DMPG 9:1; DPPC:Chol:DMPG 8:1:1; DMPC:DSPE-PEG-2000 95:5;
DMPC:Chol:DSPE-PEG-2000 90:10:05; DMPC/DMPG 7:3; DPPC/DMPG 7:3; or
DPPC/DMPG 9:1.

13. The method of claim 1, wherein the liposome comprises lipids of Formula I: 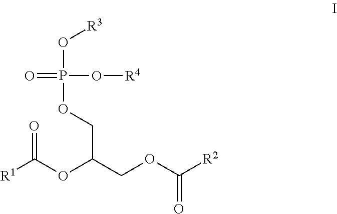
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 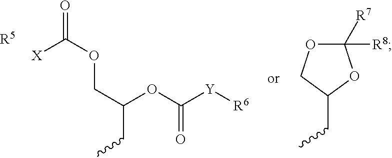
R⁴ is H or a pharmaceutically acceptable cation, wherein incorporation of said pharmaceutically acceptable cation results in a salt, e.g., a monomeric salt, a dimeric salt, a trimeric salt, or a multimeric salt;
R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁷ is a C₀-C₂₀ branched or unbranched hydrocarbon possessing 0-10 double bonds, 0-10 triple bonds or a combination of 0-10 double and triple bonds;
R⁸ is H or a C₀-C₂₀ branched or unbranched hydrocarbon possessing 0-10 double bonds, 0-10 triple bonds or a combination of 0-10 double and triple bonds;
X is a direct linkage, CH₂, O or NH;
Y is a direct linkage, CH₂, O or NH; and,
each stereogenic center is independently R, S or racemic.

14. The method of claim 13, wherein the compound of Formula I is selected from one or more of:

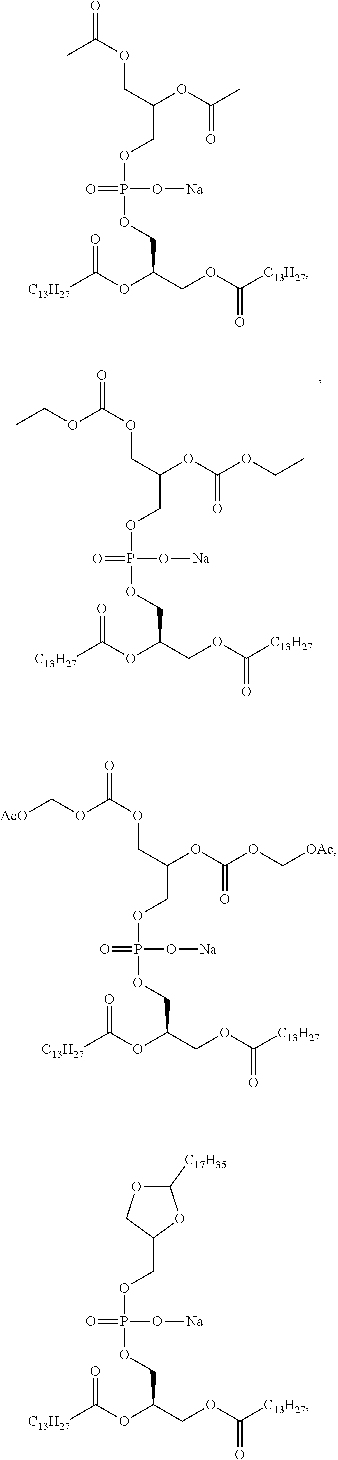

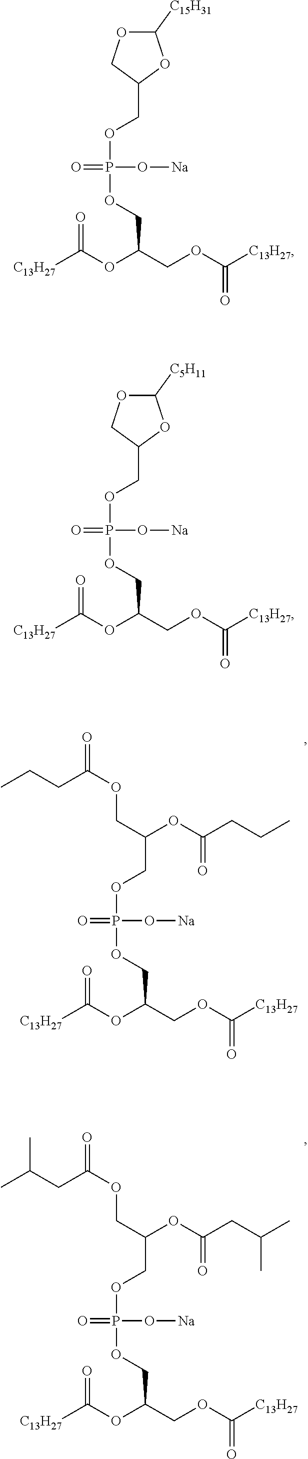

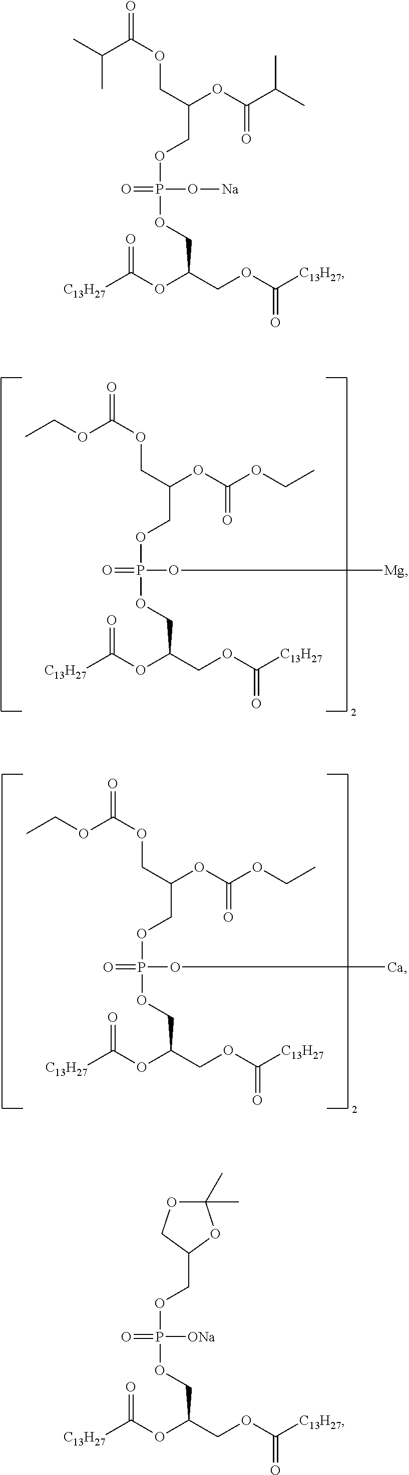

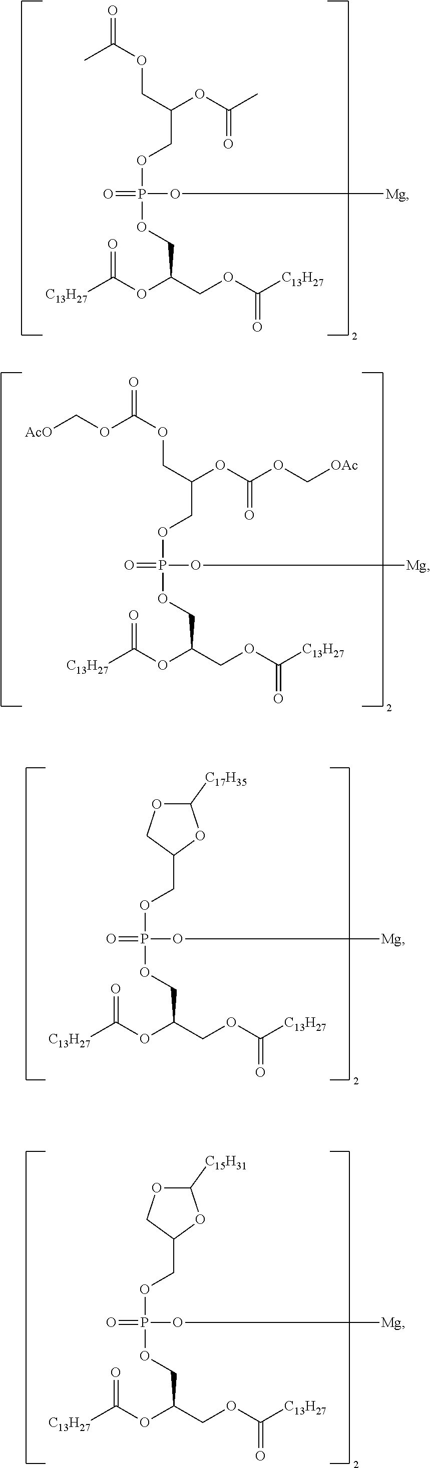

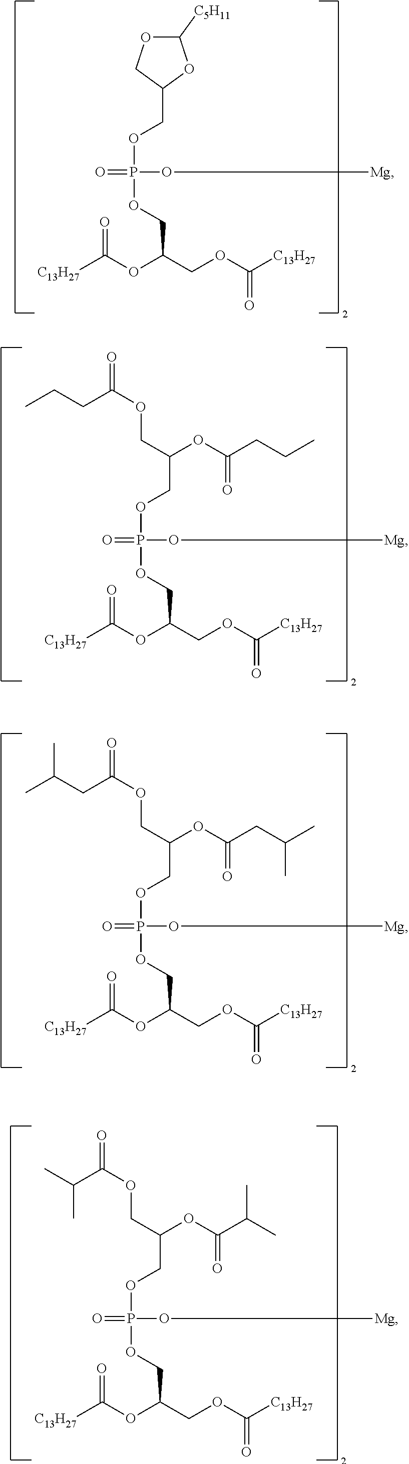

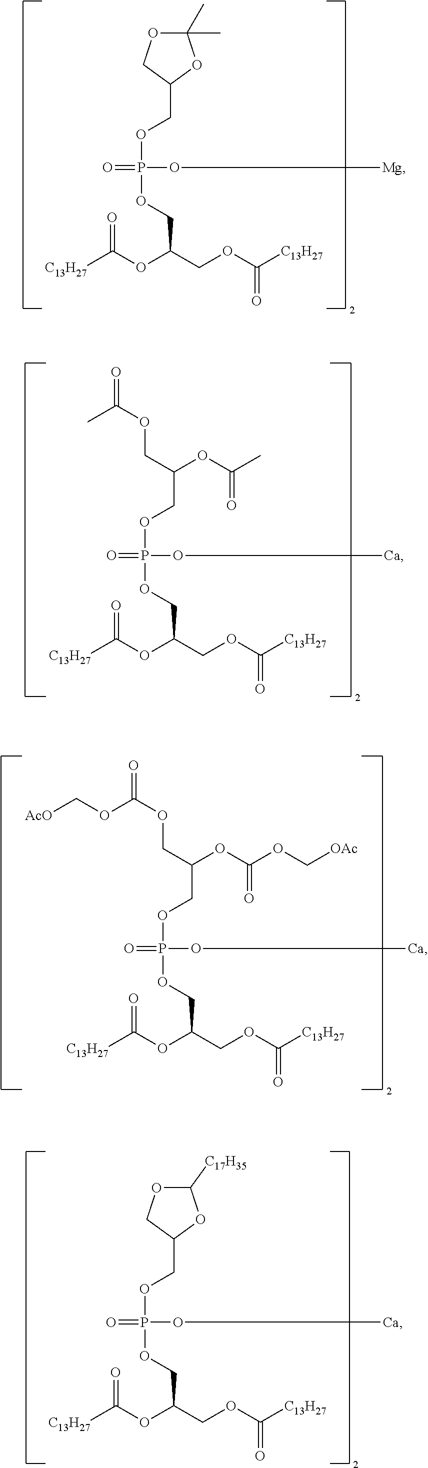

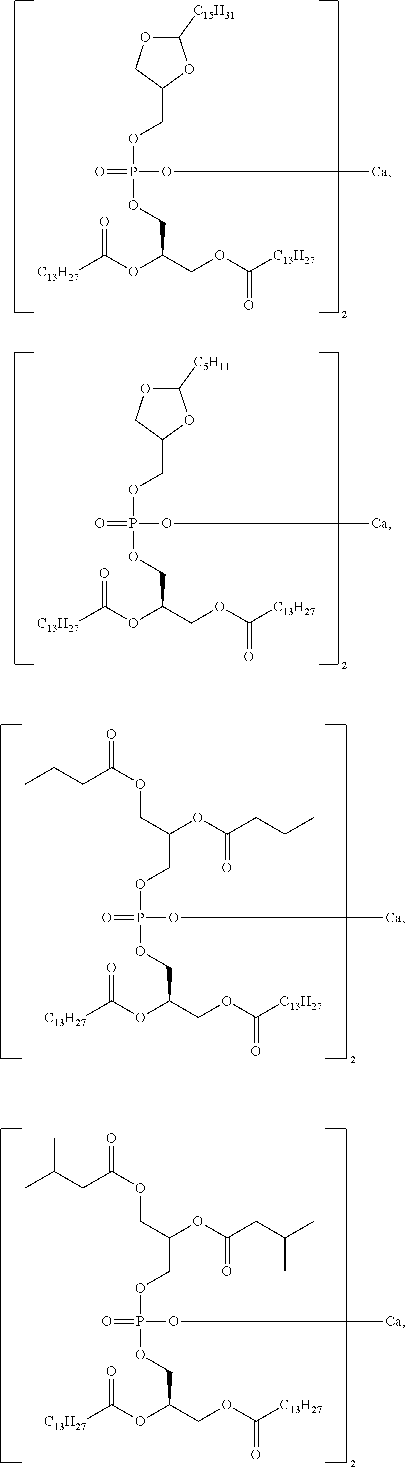

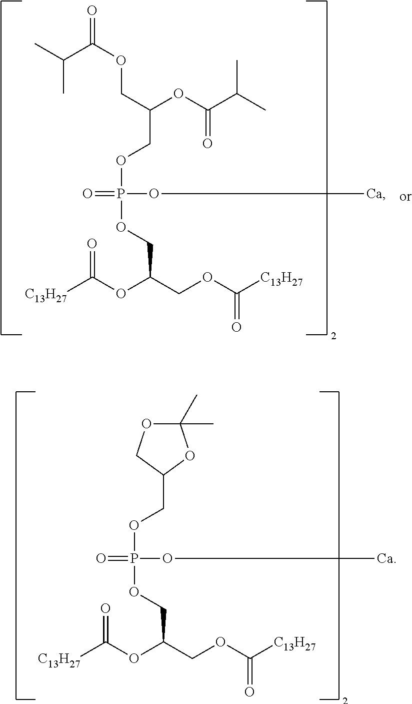

15. The method of claim 1, wherein the therapeutically effective amount of the liposomal curcumin or liposomal curcuminoids comprises 50 nM/kg, 10 to 100 nM/kg, 25 to 75 nM/kg, 10, 20, 30, 40, 50, 60, 70, 80, 90, or 100 nM/kg of body weight of the subject.

16. The method of claim 1, wherein the composition further comprises a curcumin, or a synthetic curcumin that is 85, 86, 87, 88, 89, 90, 91, 92, 93, 94, 95 or 96% pure diferuloylmethane.

17. The method of claim 1, wherein the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

18. A method for treating a malignant pleural effusion in a human patient comprising:
identifying a human patient in need of a treatment for the malignant pleural effusion; and
administering to the human patient a therapeutically effective amount of a liposomal curcumin or liposomal curcuminoids in a pleural space or cavity that is adjacent to at least one of the lung, heart, kidney, or liver, sufficient to reduce or treat the malignant pleural effusion.

19. (canceled)

20. (canceled)

21. The method of claim 18, wherein the liposomal curcumin or liposomal curcuminoids are administered at a dose of 50, 100, 125, 150, 200, 250, 300, 325, 350, 400, 450, 500, or 600 mg/m², over 2 hours, 2 to 4 hours, 2 to 6 hours, or administered for 2, 3, 4, 5, 6, or 7 hours; or
the dose is 300, 325, 350, 400, 450, 500, or 600 mg/m², over 2 hours, 2 to 4 hours, 2 to 6 hours, or administered for 2, 3, 4, 5, 6, or 7 hours; or the dose greater than 350 mg/m² over 2, 3, 4, 5, 6, 7, or 8 hours.

22. (canceled)

23. (canceled)

24. (canceled)

25. (canceled)

26. The method of claim 18, wherein the curcumin or curcuminoids are chemically synthesized curcumin or curcuminoids.

27. The method of claim 18, wherein the liposome comprises at least one of: 1,2-dimyristoyl-sn-glycero-3-phosphocholine (DMPC), 1,2-dimyristoyl-sn-glycero-3-phosphorylglycerol (DMPG), DMPC/DMPG, lysophosphatidylcholine, lauroyl-lysophosphatidylcholine, myristoyl-lysophosphatidylcholine, palmitoyl-lysophosphatidylcholine, stearoyl-lysophosphatidylcholine, arachidoyl-lysophosphatidylcholine, oleoyl-lysophosphatidylcholine, linoleoyl-lysophosphatidylcholine, linolenoyl-lysophosphatidylcholine or erucoyl-lysophosphatidylcholine, or combinations thereof.

28. The method of claim 18, wherein the liposomal curcumin or curcuminoids are curcumin/curcuminoid:liposome complex, wherein the curcumin comprises between 2 to 9 weight percent of the curcumin/curcuminoid:liposome complex, wherein the curcumin is at least one of natural or synthetic curcumin and wherein the curcumin/curcuminoid:liposome complex has a ratio of curcumin to lipid (weight to weight) of 1:7.5 to 1:10, wherein the lipid combination is selected from: DMPC; DMPC:Chol 9:1; DMPC:DMPG 9:1; DMPC:Chol:DMPG 8:1:1;
DPPC:DMPG 9:1; DPPC:Chol:DMPG 8:1:1; DMPC:DSPE-PEG-2000 95:5;
DMPC:Chol:DSPE-PEG-2000 90:10:05; DMPC/DMPG 7:3; DPPC/DMPG 7:3; or
DPPC/DMPG 9:1.

29. The method of claim 18, wherein the liposome comprises lipids of Formula I: 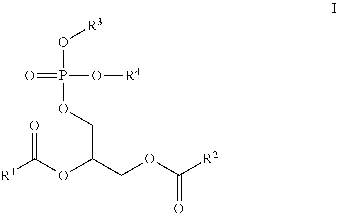
wherein,
R¹ is a C₁-C₂₀ branched or unbranched hydrocarbon possessing 0-10 double bonds, 0-10 triple bonds or a combination of 0-10 double and triple bonds;
R² is a C₁-C₂₀ branched or unbranched hydrocarbon possessing 0-10 double bonds, 0-10 triple bonds or a combination of 0-10 double and triple bonds;
R³ is 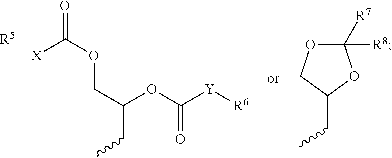
R⁴ is H or a pharmaceutically acceptable cation, wherein incorporation of said pharmaceutically acceptable cation results in a salt, e.g., a monomeric salt, a dimeric salt, a trimeric salt, or a multimeric salt;
R⁵ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁶ is a C₁-C₁₀ branched or unbranched hydrocarbon optionally substituted with one or more groups selected from OH, OAc, OMe, NH₂, NHAc, NHMe, N(Me)₂, SH, CN, COOH, CONH₂, Cl, Br and I;
R⁷ is a C₀-C₂₀ branched or unbranched hydrocarbon possessing 0-10 double bonds, 0-10 triple bonds or a combination of 0-10 double and triple bonds;
R⁸ is H or a C₀-C₂₀ branched or unbranched hydrocarbon possessing 0-10 double bonds, 0-10 triple bonds or a combination of 0-10 double and triple bonds;
X is a direct linkage, CH₂, O or NH;
Y is a direct linkage, CH₂, O or NH; and,
each stereogenic center is independently R, S or racemic.

30. The method of claim 29, wherein the compound of Formula I is selected from one or more of:

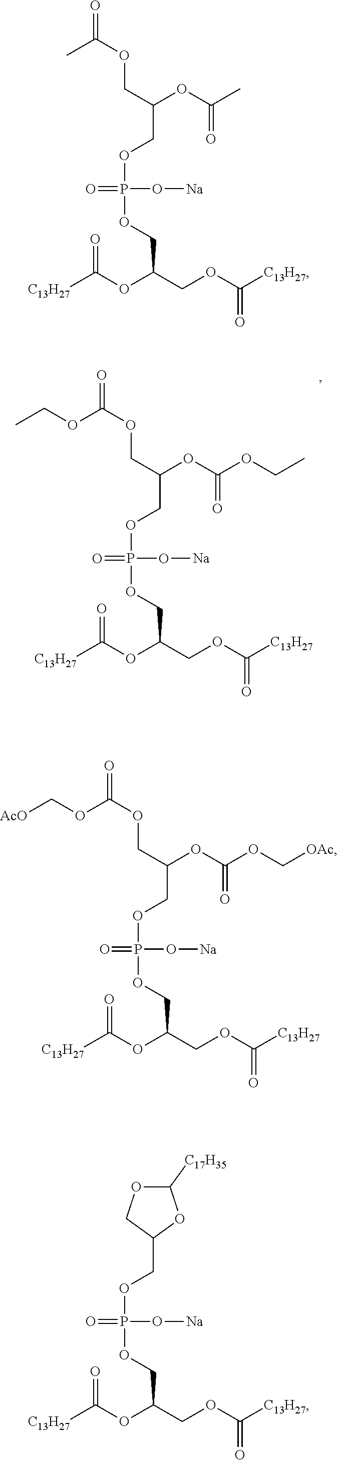

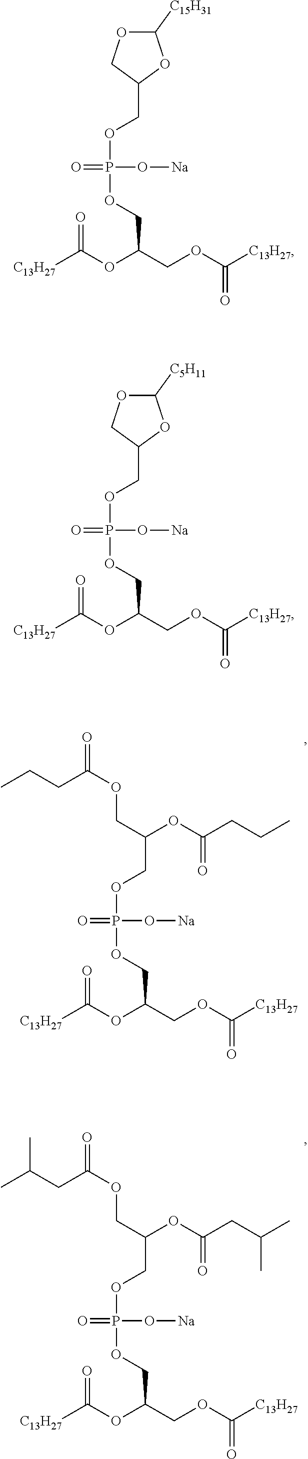

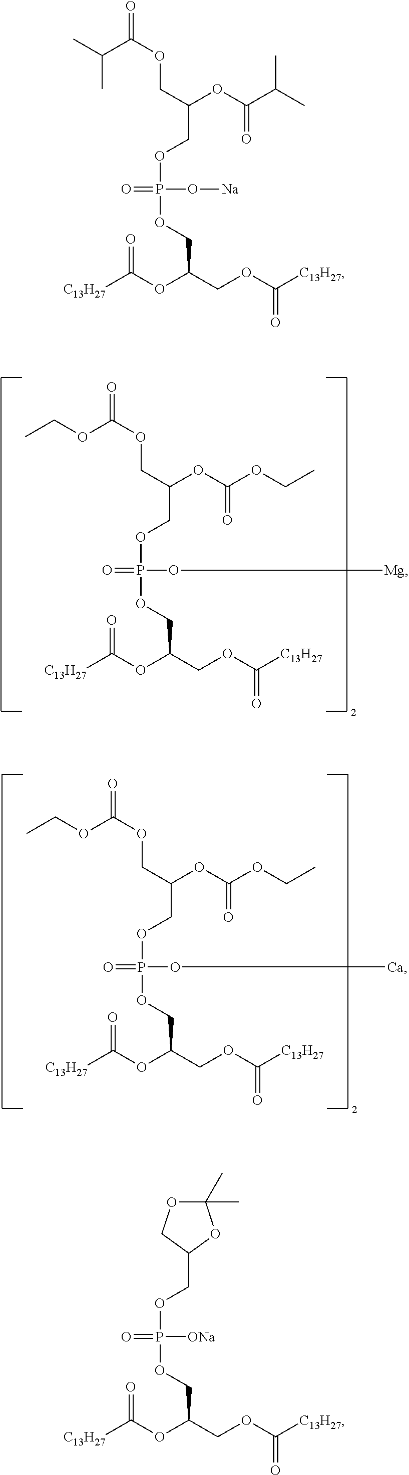

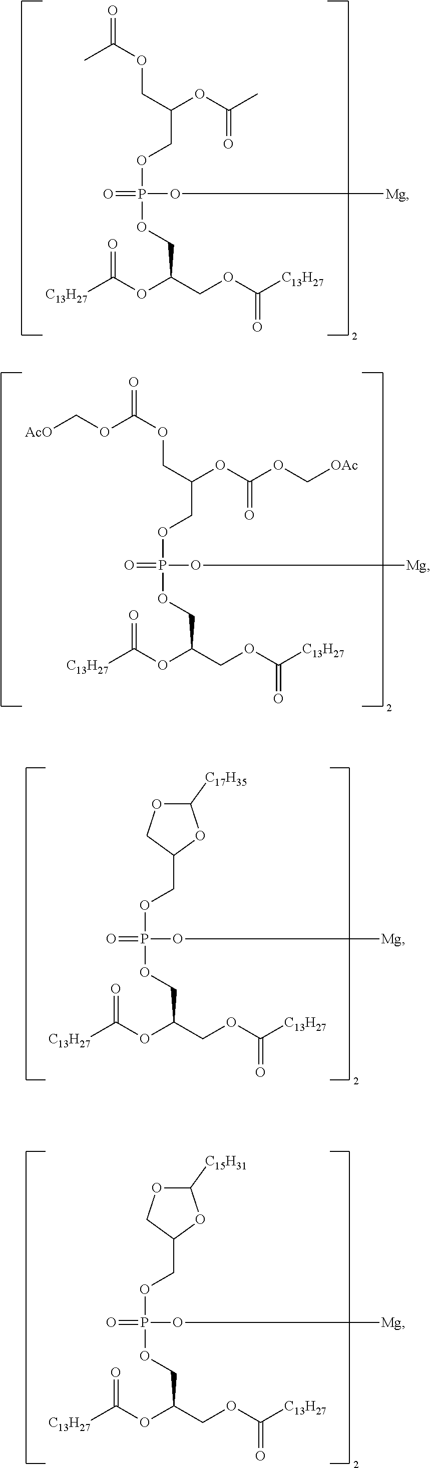

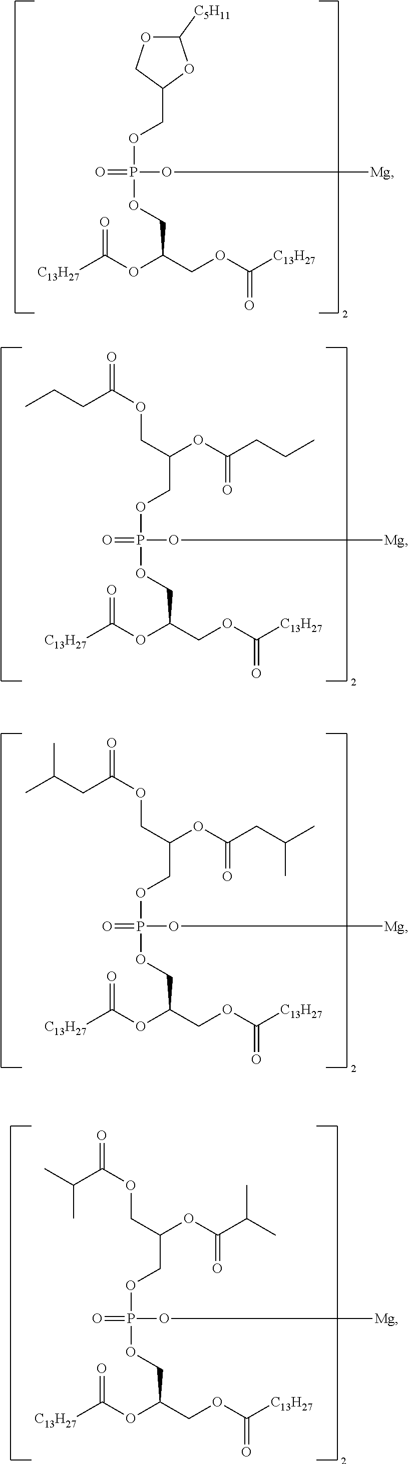

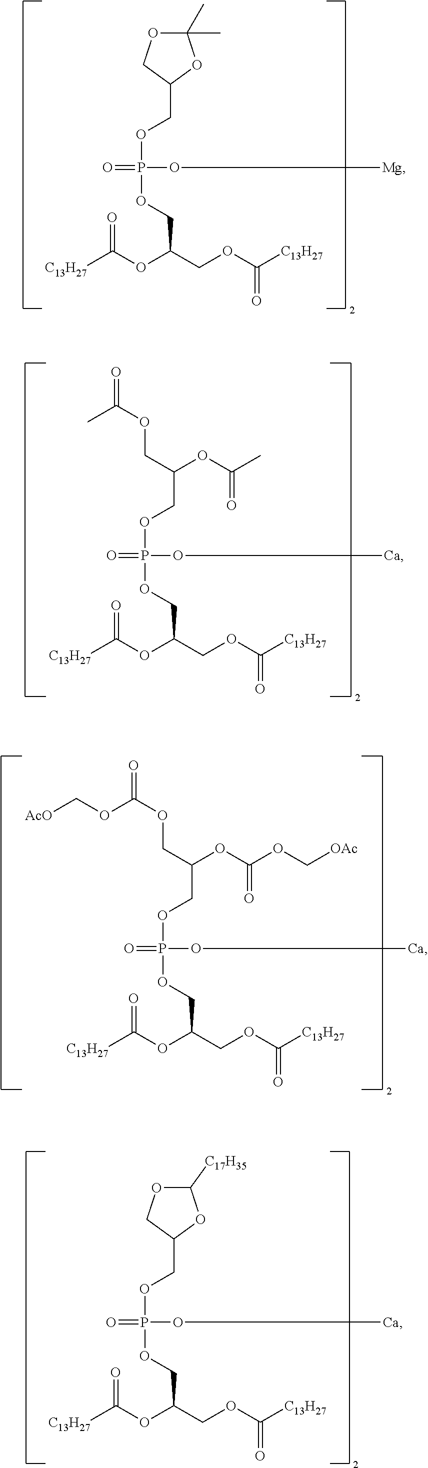

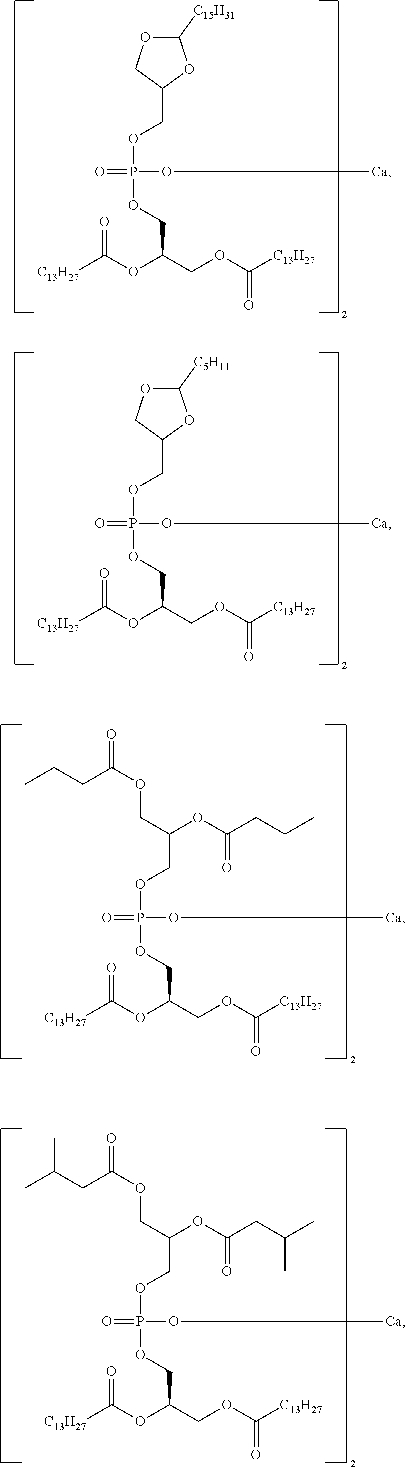

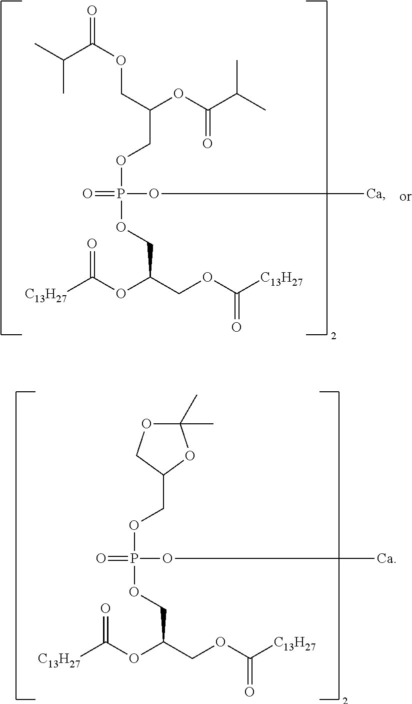

31. The method of claim 18, wherein the therapeutically effective amount comprises 50 nM/kg, 10 to 100 nM/kg, 25 to 75 nM/kg, 10, 20, 30, 40, 50, 60, 70, 80, 90, or 100 nM/kg of body weight of the subject.

32. The method of claim 18, wherein the composition further comprises a curcumin, or a synthetic curcumin that is 85, 86, 87, 88, 89, 90, 91, 92, 93, 94, 95 or 96% pure diferuloylmethane.

33. The method of claim 18, wherein the curcumin or curcuminoids are selected from at least one of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.
